(19) United States
(12) Patent Application Publication
(10) Pub. No.: US 2025/0186960 A1
(43) Pub. Date: Jun. 12, 2025

(54) BIODEGRADABLE MICROCAPSULES AND A METHOD FOR THEIR PREPARATION

(71) Applicant: XAMPLA LIMITED, Cambridge, Cambridgeshire (GB)

(72) Inventors: Lynette Anne Makins HOLLAND, Cambridge, Cambridgeshire (GB); Marc RODRIGUEZ GARCIA, Cambridge, Cambridgeshire (GB); James Ward TAYLOR, Cambridge, Cambridgeshire (GB); Juliette Marie Caroline DELARUE, Cambridge, Cambridgeshire (GB); Jack Henry Jeremy CORDREY, Cambridge, Cambridgeshire (GB); Phoebe Jane WILLIAMS, Cambridge, Cambridgeshire (GB)

(21) Appl. No.: 18/844,862

(22) PCT Filed: Mar. 8, 2023
(86) PCT No.: PCT/EP2023/055906

§ 371 Date: Sep. 6, 2024

(30) Foreign Application Priority Data
Mar. 8, 2022 (LU) 501618

(51) Int. Cl. B01J 13/10; A23L 27/00; A61K 8/06; A61K 8/11; A61K 8/64; A61Q 13/00; B01J 13/22; C11D 3/00; C11D 3/50

(52) U.S. Cl. CPC B01J 13/10; A23L 27/72; A23L 27/80; A61K 8/068; A61K 8/11; A61K 8/645; A61Q 13/00; B01J 13/22; C11D 3/001; C11D 3/505

(57) ABSTRACT

The present invention relates to a method for preparing a biodegradable microcapsule and to a method for preparing a biodegradable microcapsule composition. The present invention also relates to the biodegradable microcapsule and the biodegradable microcapsule composition per se. The present invention also relates to uses of the biodegradable microcapsules and to methods involving the biodegradable microcapsule, including to prepare a consumer product. The present invention also relates to the consumer product per se.

### FIELD OF THE INVENTION

The present invention relates to a method for preparing a biodegradable microcapsule and to a method for preparing a biodegradable microcapsule composition. The present invention also relates to the biodegradable microcapsule and the biodegradable microcapsule composition per se. The present invention also relates to uses of the biodegradable microcapsules and to methods involving the biodegradable microcapsule, including to prepare a consumer product. The present invention also relates to the consumer product per se.

### BACKGROUND

Fragrance materials and flavour materials are employed in numerous consumer products to impart a particular sensory experience to the user, e.g. fragranced fabric conditioners for imparting a fragrance to washed laundry, or flavoured mouthwashes for freshening breath. However, most fragrance/flavour materials are volatile compounds and are unstable under conventional processing conditions, meaning that their effective incorporation into consumer products can be problematic.

Encapsulation techniques, which involve embedding the fragrance/flavour material in an external matrix, can be used to protect the fragrance/flavour material from its external surroundings (e.g. exposure to chemicals, air, light etc.). This allows the shelf life of the fragrance/flavour material to be prolonged such that it can be released when needed (e.g. upon breaking the encapsulate).

Complex coacervation is one such encapsulation technique, and is based upon the electrostatic interactions between two polymers of opposite charge. However, the processing conditions required, typically high temperatures and long reaction times, mean that whilst some fragrance/flavour materials will be successfully encapsulated, the more volatile species will be lost during the process. These conventional complex coacervation techniques also normally require a subsequent covalent cross-linking modification to be made to the coacervate in order to ensure its mechanical integrity.

Other encapsulation techniques available commonly rely on the use of synthetic polymers to form a protective shell around the active ingredient. Similar problems with the encapsulation of highly volatile species are also encountered with these techniques. Additionally, synthetic polymers may not be suitable as carriers in pharmaceutical and food applications or may have limited permitted exposure levels in cosmetic applications. Furthermore, synthetic polymer shell materials can lead to the formation of microplastics, which are detrimental to the environment.

Accordingly, there exists a need for a process to prepare biodegradable microcapsules, which can encapsulate fragrance materials and/or flavour materials having a broad range of volatilities and release them when required.

Encapsulation techniques exist which use natural or naturally derived polymers, for example polysaccharides or proteins. However, the resulting encapsulates are can be highly water soluble, for example starch encapsulates, which are unable to keep their cargoes encapsulated in aqueous consumer products.

Other encapsulates, for example protein-based encapsulates, require substantial cross-linking of the shell in order to be able to keep their cargoes in aqueous consumer products to the detriment of the shell biodegradability. For example, WO 2020/209909 claims biodegradable shells of bio-polymers such as protein which may be denatured and do not go through a gelation process. These proteins are then also covalently cross-linked with two or more cross-linking agents. This results in microcapsules with poor biodegradability. There exists a need for encapsulation techniques using protein materials that are biodegradable and largely not covalently modified, but instead use a physical gelation process to obtain the desired mechanical integrity.

Encapsulation techniques exist which use silk proteins as the shell material, however these materials are not desirable due to their animal origin. In addition, animal proteins are chemically and physical different from plant proteins in that they are typically smaller and more soluble. US 2015/202304 describes a process for preparing silk proteins for an encapsulation process wherein the dried silk fibroin is solubilised in aqueous LiBr solution and then dialysed to remove the LiBr and produce a silk protein solution for an emulsion-based encapsulation process. When plant-based proteins are added to an aqueous LiBr solution they do not solubilise, but form a solid mass that cannot be used in an emulsion-based gelation encapsulation process. There remains a need for a process wherein plant-based proteins can be effectively solubilised so that they can be used in an emulsion-based gelation encapsulation process.

### SUMMARY OF THE INVENTION

Viewed from a first aspect, the present invention provides a method for preparing a biodegradable microcapsule, comprising:
(a) emulsifying a lipophilic phase comprising at least one fragrance material or flavour material in a plant-based protein solution comprising one or more plant-based protein(s) to give a primary emulsion, wherein said lipophilic phase is immiscible with said plant-based protein solution;
(b) re-emulsifying said primary emulsion in an external phase to give a secondary emulsion, wherein said external phase is immiscible with said primary emulsion;
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase; and
(d) separating the external phase from the microcapsule;
wherein said plant-based protein solution is at a temperature above the sol-gel transition temperature of the plant-based protein(s) during steps (a) and (b).

Viewed from a further aspect, the present invention provides a biodegradable microcapsule obtained by or obtainable by the method as hereinbefore described.

Viewed from a further aspect, the present invention provides a method for preparing a biodegradable microcapsule composition, comprising:
(a) emulsifying a lipophilic phase comprising a fragrance or a mixture of fragrances in a plant-based protein solution comprising one or more plant-based protein(s), wherein said lipophilic phase is immiscible with said plant-based protein solution, to give a primary emulsion;
(b) re-emulsifying said primary emulsion in an external phase to give a secondary emulsion, wherein said external phase is immiscible with said primary emulsion; and
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase;
   wherein said plant-based protein solution is at a temperature above the sol-gel transition temperature of the plant-based protein(s) during steps (a) and (b).

Viewed from a further aspect, the present invention provides a biodegradable microcapsule composition obtained by or obtainable by the method as hereinbefore described.

Viewed from a further aspect, the present invention provides a biodegradable microcapsule comprising:
(a) a lipophilic phase comprising at least one fragrance material or flavour material; and
(b) a plant-based protein hydrogel comprising at least one plant-based protein(s);
wherein:
said plant-based protein hydrogel encapsulates said lipophilic phase;
said plant-based protein hydrogel is a covalently-modified or non-covalently modified plant-based protein hydrogel or said plant-based protein hydrogel has a coating deposited thereon; and
the biodegradation percentage based upon 02 consumption of the plant-based protein hydrogel as measured according to ISO-14851 version 2019 after 28 days is 60 to 100% based upon the ratio of the Biological Oxygen Demand (BOD) to the Theoretical Oxygen Demand.

Viewed from a further aspect, the present invention provides a composition comprising a biodegradable microcapsule as hereinbefore described and an external phase.

Viewed from a further aspect, the present invention provides a consumer product comprising a biodegradable microcapsule as hereinbefore described.

Viewed from a further aspect, the present invention provides a method of making a consumer product, comprising:
(a) preparing a biodegradable microcapsule according to the method as hereinbefore described;
(b) mixing said biodegradable microcapsule with a consumer product formulation.

Viewed from a further aspect, the present invention provides the use of a biodegradable microcapsule as hereinbefore described in a consumer product.

### Definitions

As used herein, the term “fragrance” (used interchangeably with the term “perfume”) refers to the component of a formulation that is capable of imparting or modifying the odour of a product, such as a fabric conditioner or a hair conditioner, or a substrate such as fabric or hair. A fragrance is typically used to impart an overall pleasant odour or odour profile to a product either to provide a pleasurable experience, such as a Fine Fragrance, or to provide sensory cues as to the product's benefit and function, such as a calming effect for a lavender sleep aid, the idea of cleanliness for a laundry product, or to mask an unpleasant odour, such as in an insect repellent product. A “fragrance” may be composed of one or more components that can be a single chemical entity, referred to herein as a “fragrance material” (used interchangeably with the term “perfume material”), or a mixture of different “fragrance materials”. Fragrance materials can be created by either synthetic processes or extracted from nature, particularly plants, to obtain naturally occurring plant essential oils and plant extracts such as orange oil. Fragrance materials created by synthetic processes can be either new-to-the-world chemicals or nature-identical fragrance materials. Synthetic and naturally derived fragrance materials can then be blended into fragrances by skilled perfumers, also called noses, for use in consumer products. Fragrance materials can be obtained from specialist fragrance suppliers, known as fragrance houses, as individual chemicals, natural blends or as proprietary specialty blends where the full composition is not disclosed. The individual fragrance materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994) and “Perfume and Flavour Materials of Natural Origin”, S. Arctander, Ed., Elizabeth, N.J., 1960. It will be understood that for the purposes of this invention, a “fragrance material” includes a pro-fragrance such as an acetal pro-fragrance, ketal pro-fragrance, ester pro-fragrance, hydrolysable inorganic-organic pro-fragrance, and combinations thereof. The fragrance materials may be released from the pro-fragrances in a number of ways, for example, by hydrolysis, or by a shift in an equilibrium reaction, or by a pH-change, or by enzymatic release, or by UV-radiation.

A fragrance material can be described in terms of its odour strength, detection threshold, odour saturation and its character. In fragrance encapsulates it is preferable to use fragrance materials with a low odour detection threshold and a high strength so as to maximise the noticeability of even small levels of fragrance encapsulated and released.

In order to impart an odour, a fragrance material must be volatile, even if only to a small degree, as it is necessary for the molecule to be airborne to enter the nose, where it gets attached to specific neuroreceptors and elicits a signal within the olfactory system. Fragrance materials can be classified according to their volatility. Preferably fragrance materials are liquid at 20° C. and atmospheric pressure but occasionally they may be solid and can be blended with other liquid fragrance materials or solvents. Typically, the fragrance industry refers to the volatility and substantivity by loosely categorising materials into one of 3 categories: base notes for the least volatile and most substantive, heart notes for those of moderate volatility and substantivity and top notes for the most volatile and least substantive. This is based on the odour perception of the materials and is quite subjective. One way of objectively classifying the volatility of fragrance materials is according to their vapour pressure.

As used herein, the term “vapour pressure” means the partial pressure in air at a defined temperature (e.g., 25° C.) and standard atmospheric pressure (760 mmHg) for a given chemical species. It defines a chemical species' affinity for the gas phase rather than the liquid or solid state. The higher the vapour pressure the greater the proportion of the material that will, at equilibrium, be in a closed headspace. It is also related to the rate of evaporation of a fragrance material which is defined in an open environment where the material is leaving the system. The vapour pressure can be readily determined according to the reference program ACD/Percepta Desktop Software, Version 14.0 (Build: Aug. 26, 2021), Advanced Chemistry Development, Inc (ACD/Labs), Toronto, Canada, www.acdlabs.com.

A physical parameter that is relevant to the encapsulation of a fragrance material is its hydrophobicity, which may be defined in terms of its partition coefficient P. As used herein, the term “partition coefficient” refers to the ratio between the equilibrium concentration of that substance in n-octanol and in water, and is a measure of the differential solubility of said substance between these two solvents. As used herein, the term “log P” refers to the logarithm to the base 10 of the partition coefficient P. The log P can be readily determined according to the reference program ACD/Percepta Desktop Software, Version 14.0 (Build: Aug. 26, 2021), Advanced Chemistry Development, Inc (ACD/Labs), Toronto, Canada, www.acdlabs.com Log P values are predicted from the SMILE string of fragrance materials molecules. Three different types of log P values can be selected from the software. The log P Classic is based on an algorithm which takes into account a database of experimental log P values while using the principal of isolating carbons. The log P GALAS is based on an algorithm taking into account a database of a training set of compounds as well as adjusting the values with data from structurally close compounds. The consensus log P is a model based on the previous two algorithms, which can be expressed as: consensus log P=a×log P Classic+b×log P GALAS, where a and b are coefficients of the model. The latter value, consensus log P, is the log P value indicated herein.

Another aspect that is relevant to the encapsulation of a fragrance material is its Hansen Solubility Parameters (HSPs). The term HSP refers to a solubility parameter approach proposed by Charles Hansen first used to predict polymer solubility in a given solvent as described in, The Three Dimensional Solubility Parameter and Solvent Diffusion Coefficient, by Charles Hansen, Danish Technical Press (Copenhagen, 1967), This approach has since been reapplied to many other molecules. The fragrance material (or flavour material or solvent) and its interactions with its environment are defined by 3 forces: atomic dispersion forces, molecular permanent dipole forces, and molecular hydrogen bonding forces. Materials with similar HSP parameters are more likely to be miscible. These forces can be quantified by 3 values: δD, the Hansen dispersion value which relates to the Van der Waals interactions (intermolecular forces); δP, the Hansen polarity value which relates to the dipole moment (electrical charges); and δH, the Hansen Hydrogen-bonding (“h-bonding”) value. The solubility parameter δ (MPa²) is defined as δ²=δ_{D}²+δ_{P}²+δ_{H}²=E/V, where E is the cohesive energy of a solvent and V is the molar volume. The HSP values for a given material can be obtained from the HSPiP (Hansen Solubility Parameters in Practice) software available from www.hansen-solubility.com through two main different ways. Those values can either be retrieved from the Master Dataset, which comprises over 20,000 materials, by searching by name or CAS number; or be predicted by entering the SMILE string of a given molecule in the DIY section of the software, using the Y-MB (Yamamoto-Molecular Breaking) method. Furthermore, the determination of the HSP sphere relative to a given fragrance material is a good way to predict the solubility preferences within a blend of fragrance materials. The radius Ro of the HSP sphere is defined as Ro=Ra/RED, where Ra is the HSP distance between two molecules (1 and 2) expressed by: Ra²=4 (δ_{D1}−δ_{D2})²+(δ_{P1}−δ_{P2})²+(δ_{H1}−δ_{H2})², and RED is the Relative Energy Difference. This RED value can also be extracted from or predicted through the HSPiP software, and a good solvent for a given material should exhibit a RED value lower or equal to 1, whereas a solvent displaying a RED value greater than 1 should be considered as a bad solvent for the given material.

A fragrance material may be selected from an alcohol, an aldehyde, a ketone, an ester, an ether, an acetate, an alkene, a nitrile, a nitrogenous heterocyclic compound, a sulfurous heterocyclic compound, and a Schiff base.

Preferred aldehyde fragrance materials include, without limitation, alpha-amylcinnamaldehyde, anisic aldehyde, decyl aldehyde, lauric aldehyde, methyl n-nonyl acetaldehyde, methyl octyl acetaldehyde, nonylaldehyde, benzenecarboxaldehyde, neral, geranial, 1,1-diethoxy-3,7-dimethylocta-2,6-diene, 4-isopropylbenzaldehyde, 2,4-dimethyl-3-cyclohexene-1-carboxaldehyde, alpha-methyl-p-isopropyldihydrocinnamaldehyde, 3-(3-isopropylphenyl) butanal, alpha-hexylcinnamaldehyde, 7-hydroxy-3,7-dimethyloctan-1-al, 2,4-dimethyl-3-cyclohexene-1-carboxaldehyde, octyl aldehyde, phenylacetaldehyde, 2,4-dimethyl-3-cyclohexene-1-carboxaldehyde, hexanal, 3,7-dimethyloctanal, 6,6-dimethylbicyclo[3.1.1]hept-2-ene-2-butanal, nonanal, octanal, 2-nonenal undecenal, 2-methyl-4-(2,6,6-trimethyl-1-cyclohexenyl-1)-2-butenal, 2,6-dimethyloctanal, 3-(p-isopropylphenyl)propionaldehyde, 3-phenyl-4-pentenal citronellal, o/p-ethyl-alpha, alpha, 9-decenal, dimethyldihydrocinnamaldehyde, p-isobutyl-alphamethylydrocinnamaldehyde, cis-4-decen-1-al, 2,5-dimethyl-2-ethenyl-4-hexenal, trans-2-methyl-2-butenal, 3-methylnonanal, alpha-sinensal, 3-phenylbutanal, 2,2-dimethyl-3-phenylpropionaldehyde, m-tertbutyl-alpha-methyldihydrocinnamic aldehyde, geranyl oxyacetaldehyde, trans-4-decen-1-al, methoxycitronellal, and mixtures thereof.

Preferred ester fragrance materials include, without limitation, allyl cyclohexane-propionate, allyl heptanoate, allyl amyl glycolate, allyl caproate, amyl acetate (n-pentyl acetate), amyl propionate, benzyl acetate, benzyl propionate, benzyl salicylate, cis-3-hexenylacetate, citronellyl acetate, citronellyl propionate, cyclohexyl salicylate, dihydro isojasmonate, dimethyl benzyl carbinyl acetate, ethyl acetate, ethyl acetoacetate, ethyl butyrate, ethyl-2-methyl butryrate, ethyl-2-methyl pentanoate, fenchyl acetate (1,3,3-trimethyl-2-norbornanyl acetate), tricyclodecenyl acetate, tricyclodecenyl propionate, geranyl acetate, cis-3-hexenyl isobutyrate, hexyl acetate, cis-3-hexenyl salicylate, n-hexyl salicylate, isobornyl acetate, linalyl acetate, para-tertiary-butyl cyclohexyl acetate, (−)-L-menthyl acetate, ortho-tertiary-butylcyclohexyl acetate, methyl benzoate, methyl dihydro isojasmonate, alpha-methylbenzyl acetate, methyl salicylate, 2-phenylethyl acetate, prenyl acetate, cedryl acetate, cyclabute, phenethyl phenylacetate, terpinyl formate, citronellyl anthranilate, ethyl tricyclo[5.2.1.0-2,6]decane-2-carboxylate, n-hexyl ethyl acetoacetate, 2-tertbutyl-4-methyl cyclohexyl acetate, formic acid, 3,5,5-trimethylhexyl ester, phenethyl crotonate, cyclogeranyl acetate, geranyl crotonate, ethyl geranate, geranyl isobutyrate, 3,7-dimethyl-ethyl 2-nonynoate-2,6-octadienoic acid methyl ester, citronellyl valerate, 2-hexenylcyclopentanone, cyclohexyl anthranilate, L-citronellyl tiglate, butyl tiglate, pentyl tiglate, geranyl caprylate, 9-decenyl acetate, 2-isopropyl-5-methylhexyl-1 butyrate, n-pentyl benzoate, 2-methylbutyl benzoate (and mixtures thereof with pentyl benzoate), dimethyl benzyl carbinyl propionate, dimethyl benzyl carbinyl acetate, trans-2-hexenyl salicylate, dimethyl benzyl carbinyl isobutyrate, 3,7-dimethyloctyl formate, rhodinyl formate, rhodinyl isovalerate, rhodinyl acetate, rhodinyl butyrate, rhodinyl propionate, cyclohexylethyl acetate, neryl butyrate, tetrahydrogeranyl butyrate, myrcenyl acetate, 2,5-dimethyl-2-ethenylhex-4-enoic acid, methyl ester, 2,4-dimethylcyclohexane-1-methyl acetate, ocimenyl acetate, linalyl isobutyrate, 6-methyl-5-heptenyl-1 acetate, 4-methyl-2-pentyl acetate, n-pentyl 2-methylbutyrate, propyl acetate, isopropenyl acetate, isopropyl acetate, 1-methylcyclohex-3-ene-carboxylic acid, methyl ester, propyl tiglate, propyl/isobutyl cyclopent-3-enyl-1-acetate (alphavinyl), butyl 2-furoate, ethyl 2-pentenoate, (E)-methyl 3-pentenoate, 3-methoxy-3-methylbutyl acetate, n-pentyl crotonate, n-pentyl isobutyrate, propyl formate, furfuryl butyrate, methyl angelate, methyl pivalate, prenyl caproate, furfuryl propionate, diethyl malate, isopropyl 2-methylbutyrate, dimethyl malonate, bornyl formate, styralyl acetate, 1-(2-furyl)-1-propanone, 1-citronellyl acetate, 3,7-dimethyl-1,6-nonadien-3-yl acetate, neryl crotonate, dihydromyrcenyl acetate, tetrahydromyrcenyl acetate, lavandulyl acetate, 4-cyclooctenyl isobutyrate, cyclopentyl isobutyrate, 3-methyl-3-butenyl acetate, allyl acetate, geranyl formate, cis-3-hexenyl caproate, and mixtures thereof.

Preferred alcohol fragrance materials include, without limitation, benzyl alcohol, beta-gamma-hexenol (2-hexen-1-ol), cedrol, citronellol, cinnamic alcohol, p-cresol, cumic alcohol, dihydromyrcenol, 3,7-dimethyl-1-octanol, dimethyl benzyl carbinol, eucalyptol, eugenol, fenchyl alcohol, geraniol, hydratopic alcohol, isononyl alcohol (3,5,5-trimethyl-1-hexanol), linalool, methyl chavicol (estragole), methyl eugenol (eugenyl methyl ether), nerol, 2-octanol, patchouli alcohol, phenyl hexanol (3-methyl-5-phenyl-1-pentanol), phenethyl alcohol, alpha-terpineol, tetrahydrolinalool, tetrahydromyrcenol, 4-methyl-3-decen-5-ol, 1-3,7-dimethyloctane-1-ol, 2-(furfuryl-2)-heptanol, 6,8-dimethyl-2-nonanol, ethyl norbornyl cyclohexanol, beta-methyl cyclohexane ethanol, 3,7-dimethyl-(2),6-octen(adien)-1-ol, trans-2-undecen-1-ol, 2-ethyl-2-prenyl-3-hexenol, isobutyl benzyl carbinol, dimethyl benzyl carbinol, ocimenol, 3,7-dimethyl-1,6-nonadien-3-ol (cis & trans), tetrahydromyrcenol, alpha-terpineol, 9-decenol-1, 2-(2-hexenyl)-cyclopentanol, 2,6-dimethyl-2-heptanol, 3-methyl-1-octen-3-ol, 2,6-dimethyl-5-hepten-2-ol, 3,7,9-trimethyl-1,6-decadien-3-ol, 3,7-dimethyl-6-nonen-1-ol, 3,7-dimethyl-1-octyn-3-ol, 2,6-dimethyl-1,5,7-octatrienol-3, dihydromyrcenol, 2,6,-trimethyl-5,9-undecadienol, 2,5-dimethyl-2-propylhex-4-enol-1, (Z)-3-hexenol, o,m,p-methyl-phenylethanol, 2-methyl-5-phenyl-1-pentanol, 3-methylphenethyl alcohol, para-methyl dimethyl benzyl carbinol, methyl benzyl carbinol, p-methylphenylethanol, 3,7-dimethyl-2-octen-1-ol, 2-methyl-6-methylene-7-octen-4-ol, and mixtures thereof.

Preferred ketone fragrance materials include, without limitation, oxacycloheptadec-10-en-2-one, benzylacetone, benzophenone, L-carvone, cis-jasmone, 4-(2,6,6-trimethyl-3-cyclohexen-1-yl)-but-3-en-4-one, ethyl amyl ketone, alpha-ionone, ionone beta, ethanone, octahydro-2,3,8,8-tetramethyl-2-acetonaphthalene, alpha-irone, 1-(5,5-dimethyl-1-cyclo-hexen-1-yl)-4-penten-1-one, 3-nonanone, ethyl hexyl ketone, menthone, 4-methyl-acetophenone, gamma-methyl ionone, methyl pentyl ketone, methyl heptenone (6-methyl-5-hepten-2-one), methyl heptyl ketone, methyl hexyl ketone, delta muscenone, 2-octanone, 2-pentyl-3-methyl-2-cyclopenten-1-one, 2-heptylcyclopentanone, alpha-methylionone, 3-methyl-2-(trans-2-pentenyl)-cyclopentenone, octenyl cyclopentanone, n-amylcyclopentenone, 6-hydroxy-3,7-dimethyloctanoic acid lactone, 2-hydroxy-2-cyclohexen-1-one, 3-methyl-4-phenyl-3-buten-2-one, 2-pentyl-2,5,5-trimethylcyclopentanone, 2-cyclopentylcyclopentanol-1, 5-methylhexan-2-one, gamma-dodecalactone, delta-dodecalactone, gamma-nonalactone, delta-nonalactone, gamma-octalactone, delta-undecalactone, gamma-undecalactone, alpha damascone, beta damascone, gamma damascone, delta damascone and mixtures thereof.

Preferred ether fragrance materials include, without limitation, diphenyl oxide, p-cresyl methyl ether, 4,6,6,7,8,8-hexamethyl-1,3,4,6,7,8-hexahydro-cyclopenta(G)-2-benzopyran, beta-naphthyl methyl ether, methyl isobutenyl tetrahydropyran, 5-acetyl-1,1,2,3,3,6-hexamethylindan (phantolide), 7-acetyl-1,1,3,4,4,6-hexamethyltetralin (tonalid), 2-phenylethyl-3-methylbut-2-enyl ether, ethyl geranyl ether, phenylethyl isopropyl ether, and mixtures thereof.

Preferred alkene fragrance materials include, without limitation, allo-ocimene, camphene, beta-caryophyllene, cadinene, diphenylmethane, d-limonene, lymolene, beta-myrcene, para-cymene, 2-alpha-pinene, beta-pinene, alpha-terpinene, gamma-terpinene, terpineolene, 7-methyl-3-methylene-1,6-octadiene, and mixtures thereof.

Preferred nitrile fragrance materials include, without limitation, 3,7-dimethyl-6-octenenitrile, 3,7-dimethyl-2(3), 6-nonadienenitrile, (2E,6Z)-2,6-nonadienenitrile, n-dodecane nitrile, and mixtures thereof.

Preferred Schiff base fragrance materials include, without limitation, citronellyl nitrile, nonanal/methyl anthranilate, N-octylidene-anthranilic acid methyl ester, hydroxycitronellal/methyl anthranilate, cyclamen aldehyde/methyl anthranilate, methoxyphenylpropanal/methyl anthranilate, ethyl p-aminobenzoate/hydroxycitronellal, citral/methyl anthranilate, 2,4-dimethylcyclohex-3-enecarbaldehyde methyl anthranilate, hydroxycitronellal-indole, and mixtures thereof.

As used herein, the term “low volatility fragrance material” is classified as a fragrance material having a vapour pressure less than 0.001 Torr (0.000133 kPa) at 25° C.

As used herein, the term “moderate volatility fragrance material” is classified as a fragrance material having a vapour pressure greater than or equal to 0.001 Torr (0.000133 kPa) at 25° C. but less than 0.1 Torr (0.0133 kPa) at 25° C.

As used herein, the term “high volatility fragrance material” is classified as one having a vapour pressure greater than or equal to 0.1 Torr (0.0133 kPa) at 25° C.

As used herein, the term “flavour” refers to the component of a formulation that is capable of imparting or modifying the taste and smell of a product, such as a toothpaste or foodstuff. A flavour is typically used to impart an overall pleasant taste and smell, or a taste and smell profile, to a product either to simply provide a pleasurable experience, such as in a foodstuff, or to mask an unpleasant taste or smell, such as in a medicine. A flavour or flavour material can be described in terms of its aroma strength, detection threshold and quality. A “flavour” may be composed of one or more components that can be a single chemical entity, referred to herein as a “flavour material”, or a mixture of different “flavour materials”. Flavour materials can be created by either synthetic processes or extracted from nature, particularly from plants, to create naturally occurring plant and animal oils and exudate, such as vanilla. Synthetic and naturally derived flavour materials can then be blended into flavours by skilled flavourists for use in consumer products. Flavour materials can be obtained from specialist flavour suppliers, known as flavour houses, as individual chemicals, natural blends or as proprietary specialty blends where the full composition is not disclosed. The individual flavour materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994); “Perfume and Flavour Materials of Natural Origin”, S. Arctander, Ed., Elizabeth, N.J., 1960; and “Flavourings”, E. Ziegler and H. Ziegler (ed.), Wiley-VCH Weinheim, 1998. It will be understood that flavours can be volatile or have volatile components which are detected by the nose in the same way as fragrances. As such, flavour materials can also be classified according to their physical characteristics, such as volatility and hydrophobicity, using the methods described above for fragrance materials. Flavour materials can also be described according to their Hansen Solubility Parameters using the methods described above for fragrance materials.

Sources of flavour materials include essential oils, concretes, absolutes, resins, resinoids, balsams, and tinctures. Preferred flavour materials include anise oil, ethyl-2-methyl butyrate, vanillin, cis-3-heptenol, cis-3-hexenol, trans-2-heptenal, butyl valerate, 2,3-diethyl pyrazine, methylcyclo-pentenolone, benzaldehyde, valerian oil, 3,4-dimeth-oxyphenol, amyl acetate, amyl cinnamate, y-butyryl lactone, trimethyl pyrazine, phenyl acetic acid, isovaleraldehyde, ethyl maltol, ethyl vanillin, ethyl valerate, ethyl butyrate, cocoa extract, coffee extract, peppermint oil, spearmint oil, clove oil, anethol, cardamom oil, wintergreen oil, cinnamic aldehyde, ethyl-2-methyl valerate, g-hexenyl lactone, 2,4-decadienal, 2,4-heptadienal, methyl thiazole alcohol (4-methyl-5-b-hydroxyethyl thiazole), 2-methyl butanethiol, 4-mercapto-2-butanone, 3-mercapto-2-pentanone, 1-mercapto-2-propane, benzaldehyde, furfural, furfuryl alcohol, 2-mercapto propionic acid, alkyl pyrazine, methyl pyrazine, 2-ethyl-3-methyl pyrazine, tetramethyl pyrazine, polysulfides, dipropyl disulphide, methyl benzyl disulphide, alkyl thiophene, 2,3-dimethyl thiophene, 5-methyl furfural, acetyl furan, 2,4-decadienal, guiacol, phenyl acetaldehyde, b-decalactone, d-limonene, acetoin, amyl acetate, maltol, ethyl butyrate, levulinic acid, piperonal, ethyl acetate, n-octanal, n-pentanal, n-hexanal, diacetyl, monosodium glutamate, monopotassium glutamate, sulfur-containing amino acids, e.g., cysteine, 2-methylfuran-3-thiol, 2-methyldihydrofuran-3-thiol, 2,5-dimethylfuran-3-thiol, tetramethyl pyrazine, propylpropenyl disulphide, propylpropenyl trisulfide, diallyl disulphide, diallyl trisulfide, dipropenyl disulphide, dipropenyl trisulfide, 4-methyl-2-[(methylthio)-ethyl]-1,3-dithiolane, 4,5-dimethyl-2-(methylthiomethyl)-1,3-dithiolane, and 4-methyl-2-(methylthiomethyl)-1,3-dithiolane, hop oils, and citrus oils such as lemon, orange, lime, grapefruit,

As used herein, the term “low volatility flavour material” is classified as a flavour material having a vapor pressure less than 0.001 Torr (0.000133 kPa) at 25° C.

As used herein, the term “moderate volatility flavour material” is classified as a flavour material having a vapour pressure greater than or equal to 0.001 Torr (0.000133 kPa) at 25° C. but less than 0.1 Torr (0.0133 kPa) at 25° C.

As used herein, the term “high volatility flavour material” is classified as a flavour material having a vapour pressure greater than or equal to 0.1 Torr (0.0133 kPa) at 25° C.

As used herein, the term “lipophilic phase” refers to a phase comprising at least one fragrance material or flavour material, which is immiscible with a plant-based protein solution as described herein.

As used herein, the term “oil-miscible solvent” refers to a substance that is capable of dissolving the at least one fragrance or flavour material.

As used herein, the term “primary emulsion” refers to a system wherein a lipophilic phase is emulsified in an immiscible aqueous plant-based protein solution.

As used herein, the term “secondary emulsion” refers to a primary emulsion, which has itself been emulsified in a further immiscible external phase.

As used herein, the term “core-shell microcapsule” refers to a microcapsule having a central core that is a lipophilic phase, wherein the lipophilic phase is surrounded by a plant-based protein hydrogel in the form of a shell.

As used herein, the term “multicore morphology” is used to describe a core-shell microcapsule wherein the lipophilic phase is in the form of a plurality of droplets.

As used herein, the term “single core morphology” is used to describe a core-shell microcapsule wherein the lipophilic phase is in the form of a single droplet.

As used herein, the term “matrix microcapsule” refers to a microcapsule comprised of a plant-based protein hydrogel matrix in which a lipophilic phase has been dispersed throughout.

As used herein, the term “standard fabric conditioner formulation” refers to a fabric conditioner having the composition described in Table 1 in the Examples section.

As used herein, the term “sol-gel transition temperature” refers to the temperature at which a plant-based protein transforms from a liquid state into a hydrogel state. Thus, at temperatures above the sol-gel transition temperature, the plant-based protein will be in a liquid state, and at temperatures below the sol-gel transition temperature the plant-based protein will be in a hydrogel state.

### DETAILED DESCRIPTION OF THE INVENTION

The present invention is directed to a method for preparing a biodegradable microcapsule, comprising:
(a) emulsifying a lipophilic phase comprising at least one fragrance material or flavour material in a plant-based protein solution comprising one or more plant-based protein(s) to give a primary emulsion, wherein said lipophilic phase is immiscible with said plant-based protein solution;
(b) re-emulsifying said primary emulsion in an external phase to give a secondary emulsion, wherein said external phase is immiscible with said primary emulsion;
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase; and
(d) separating the external phase from the microcapsule;
wherein said plant-based protein solution is at a temperature above the sol-gel transition temperature of the plant-based protein(s) during steps (a) and (b).

Any suitable plant-based proteins may be used in the present invention. In preferred methods of the present invention, the plant-based protein(s) is obtained from fava bean, mung bea, pea, rice, potato, rapeseed, lentil, chickpea, sunflower seed, pumpkin seed, flax, chia, canola, lupine, alfalfa, moringa, wheat, corn zein or sorghum; preferably the plant protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein, more preferably pea protein and/or potato protein. Such proteins are considered to be low allergenicity proteins.

Suitable plant-based proteins further include:
Brassicas: including Brassica balearica: Mallorca cabbage, Brassica carinata: Abyssinian mustard or Abyssinian cabbage, Brassica elongata: elongated mustard, Brassica fruticulosa: Mediterranean cabbage, Brassica hilarionis: St Hilarion cabbage, Brassica juncea: Indian mustard, brown and leaf mustards, Sarepta mustard, Brassica napus: rapeseed, canola, rutabaga, Brassica narinosa: broadbeaked mustard, Brassica nigra: black mustard, Brassica oleracea: kale, cabbage, collard greens, broccoli, cauliflower, kai-lan, Brussels sprouts, kohlrabi, Brassica perviridis: tender green, mustard spinach, Brassica rapa (syn. B. campestris): Chinese cabbage, turnip, rapini, komatsuna, Brassica rupestris: brown mustard, Brassica toumefortii: Asian mustard
Solanaceae: including tomatoes, potatoes, eggplant, bell and chili peppers;
cereals: including maize, rice, wheat, barley, sorghum, millet, oats, rye, triticale, fonio
pseudocereals: including amaranth (love-lies-bleeding, red amaranth, prince-of-Wales-feather), breadnut, buckwheat, chia, cockscomb (also called quail grass or soko), pitseed Goosefoot, qahiwa, quinoa and, wattleseed (also called acacia seed);
Legume: including Acacia alata (Winged Wattle), Acacia decipiens, Acacia saligna (commonly known by various names including coojong, golden wreath wattle, orange wattle, blue-leafed wattle), Arachis hypogaea (peanut), Astragalus galegiformis, Cytisus laburnum (the common laburnum, golden chain or golden rain), Cytisus supinus, Dolichios lablab (common names include hyacinth bean, lablab-bean bonavist bean/pea, dolichos bean, seim bean, lablab bean, Egyptian kidney bean, Indian bean, bataw and Australian pea), Ervum lens (Lentil), Genista tinctorial (common names include dyer's whin, waxen woad and waxen wood), Glycine max (Soybean), Lathyrus clymenum (peavines or vetchlings), Lathyrus odoratus (peavines or vetchlings), Lathyrus staivus (peavines or vetchlings), Lathyrus silvetris (peavines or vetchlings), Lotus tetragonolobus (asparagus-pea or winged pea), Lupinus albus (Lupin), Lupinus angustifolius (lupin), Lupinus luteus (Lupin), Lupinus polyphyllus (Lupin), Medicago sativa (Alfalfa), Phaseolus aureus (Mung bean), Phaseolus coccineus (Runner bean), Phaseolus nanus (Green bean/French bean), Phaseolus vulgaris (Green bean/French bean), Pisum sativum (pea), Trifolium hybridum (Clover), Trifolium pretense (Red clover), Vicia faba (Broad bean), Vicia sativa (Vetch), Vigna unguiculate (cowpea)
Non-Legumes: including: Acanshosicyos horrida (Acanshosicyos horrida), Aesculus hyppocastanum (Conker tree/Horsechestnut), Anacardium occidentale (Cashew tree), Balanites aegyptica, Bertholletia excels (Brazil nut), Beta vulgaris (Sugar beet), Brassica napus (Rapeseed), Brassica juncea (Brown mustard), Brassica nigra (Black mustard), Brassica hirta (Eurasian mustard), Cannabis sativa (marijuana), Citrullus vulgaris (Sort of watermelon), Citrus aurantiaca (Citrus), Cucurbita maxima (squash), Fagopyrum esculentum (knotweed), Gossypium barbadense (Extra-long staple cotton), Helianthus annuus (sunflower), Nicotiana sp. (Tobacco plant), Prunus avium (cherry), Prunus cerasus (Sour cherry), Prunus domestica (plum), Prunus amygdalus (almond), Ricinus communis (Caster bean/caster oil plant), Sasamum indicum (Sesame), Sinapis alba (White mustard), Terlfalrea pedata (Oyster nut).

For the avoidance of doubt, the plant-based microcapsules of the present invention do not encompass plants in their natural state, e.g. naturally formed plant cells, organelles or vesicles are not plant-based microcapsules of the present invention.

In preferred methods of the present invention, the at least one fragrance material or flavour material is selected from an alcohol, an aldehyde, a ketone, an ester, an ether, an acetate, an alkene, a nitrile, a nitrogenous heterocyclic compound, a sulfurous heterocyclic compound, and a Schiff base.

The fragrance materials and flavour materials employed in the present invention may be of natural origin (i.e. they are extracted from a natural source and are not synthetically modified in any way). Preferred fragrance materials or flavour materials of natural origin include nutmeg extract, cardamon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, jasmine extract, olibanum extract, rose extract, vanillin, coffee extract, hop oil or combinations thereof. Preferably, the fragrance materials or flavour materials of natural origin are plant-derived. The fragrance materials or flavour materials of natural origin may be used alone, or in combination, or in combination with synthetic fragrance materials.

In preferred methods of the present invention, the at least one fragrance material or flavour material has a vapour pressure of greater than or equal to 0.0001 Torr at 25° C., preferably greater than or equal to 0.001 Torr at 25° C.

In preferred methods of the present invention, the at least one fragrance material or flavour material has a log P greater than or equal to 3.0.

In preferred methods of the present invention, the at least one fragrance material or flavour material has at least two Hansen solubility parameters selected from: an atomic dispersion force (δD) from 14 to 20, a dipole moment (δP) of less than 8, and a hydrogen bonding (δH) from 2.5 to 11.

In preferred methods of the present invention, the at least one fragrance material or flavour material is part of a fragrance or flavour.

Preferably, the fragrance or flavour contains at least 20 wt % of fragrance material(s) or flavour material(s) with a vapour pressure greater than or equal to 0.001 Torr at 25° C. based upon the total weight of the fragrance or flavour, more preferably 40 wt %, more preferably 60 wt %.

Preferably, the fragrance or flavour contains at least 20 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 30 wt %, more preferably at least 50 wt %, more preferably at least 70 wt %.

Preferably, the fragrance or flavour contains at least 10 wt % of fragrance material(s) or flavour material(s) which have a biodegradation percentage based upon O₂ consumption as measured according to ISO-14851 version 2019 after 28 days of 60 to 100% based upon the ratio of the Biological Oxygen Demand (BOD) to the Theoretical Oxygen Demand, more preferably 65%, even more preferably 70 to 100%, even more preferably 75 to 100%, even more preferably 80 to 100%, even more preferably 85 to 100%, most preferably 90 to 100%. Due to their low aqueous solubility, fragrance or flavours can be added to the biodegradation test on an inert support according to the method in ISO 10634 version 2018: Water quality—Preparation and treatment of poorly water-soluble organic compounds for the subsequent evaluation of their biodegradability in an aqueous medium. In addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (δD) from 14 to 20, a dipole moment (δP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality). In preferred methods of the present invention, the fragrance or flavour comprises less than 40% wt alcohol-containing material based upon the total weight of the fragrance or flavour, more preferably less than 20% wt. In particularly preferred methods of the present invention, the fragrance or flavour does not comprise an alcohol-containing material. Without wishing to be bound by theory, it is thought that alcohols, and particularly primary alcohols having a straight chain alkyl group, can easily diffuse through the shell of a microcapsule due to their structure, meaning that they can be difficult materials to encapsulate.

Preferably, the fragrance or flavour material is of high odour impact. This is advantageous as it ensures that even low levels of fragrance are perceived when released from the microcapsules.

In preferred methods of the present invention, the plant-based protein solution comprises one or more plant-based protein(s) in a solvent system, wherein the solvent system comprises miscible co-solvents; wherein a first co-solvent increases solubility of the plant-based protein(s), and a second co-solvent decreases solubility of the plant-based protein(s).

The first co-solvent increases solubility of the plant-based protein(s). The first co-solvent may be considered a solubilising co-solvent. There may be one or more solubilising co-solvent(s) and the solubilising co-solvent(s) may fully or partially solubilise the plant-based protein(s).

Examples of solubilising co-solvents are organic acids. An organic acid is an organic compound with acidic properties. Preferably, the organic acids are sourced from natural plant-based or bio-based feed-stocks.

In preferred methods of the present invention, the first co-solvent is an organic acid. Preferably, the organic acid is acetic acid, lactic acid, formic acid, gluconic acid, propionic acid, an α-hydroxy acid, or a β-hydroxy acid. Preferred α-hydroxy acids include glycolic acid, acetic acid, lactic acid, malic acid, citric acid and tartaric acid, preferably acetic acid and lactic acid. Preferred β-hydroxy acids include β-hydroxypropionic acid, β-hydroxybutyric acid, β-hydroxy β-methylbutyric acid, 2-hydroxybenzoic acid and carnitine. In particularly preferred methods of the present invention, the organic acid is acetic acid or lactic acid.

Using an organic acid enables solubilisation of the plant protein and also allows for mild hydrolysis of the protein. For example, without wishing to be bound by theory, the solubility of plant-based proteins in organic acid is possible due to: i) the protonation of proteins and ii) the presence of an anion solvation layer which contributes to a reduction of hydrophobic interactions. Once initially dissolved in organic acid, the protonation of plant-based proteins can help to stabilise them in its non-solvent, for example water.

The second co-solvent has decreased solubility of the plant based protein(s), as compared to the first co-solvent. The second co-solvent may be considered a de-solubilising co-solvent. There may be one or more de-solubilising co-solvent(s). In preferred methods of the present invention, the second co-solvent is selected from water, ethanol, and/or ethyl acetate, more preferably water and/or ethanol, even more preferably water.

In preferred methods of the present invention, the solvent system comprises a co-solvent ratio of first co-solvent to second co-solvent of about 10-90% v/v, about 20-90% v/v, preferably about 20-80% v/v, preferably about 20-60% v/v, about 25-55% v/v, about 30-50% v/v, about 20%, about 30%, about 40% about 50% or about 60% v/v, most preferably about 30-50% v/v.

In preferred methods of the present invention, the first co-solvent is present in the plant-based protein solution at a concentration based on weight-% of equal or greater than the concentration of the protein.

In preferred methods of the present invention, the concentration of the plant-based protein(s) in the solvent system is 25-200 mg/ml, more preferably 50-150 mg/ml. The ratio of organic acid may vary depending on protein concentration, e.g. using a higher organic acid ratio with increasing protein concentration.

In preferred methods of the present invention, the degree of protein hydrolysis is controlled to modify the properties of the resultant hydrogel. For example, increasing the acid concentration present during formation will increase the degree of protein hydrolysis. Higher degree of protein hydrolysis leads to the formation of less rigid hydrogels.

In order to form the solution comprising one or more plant-based protein(s), it may be necessary to apply physical stimulus to the protein/solvent system mixture to enable dissolution of the protein. Suitable physical stimulus includes heating, ultrasonication, agitation, high-shear mixing, high-shear homogenisation or other physical techniques. A preferred technique is heating, optionally with subsequent ultrasonication.

Preferably, the protein/solvent system mixture is subjected to a physical stimulus which is heating, wherein the solution is heated to about or above 70° C. More preferably, the protein/solvent system mixture is heated to about or above 75° C., about or above 80° C., about or above 85° C. or about 90° C. Even more preferably, the protein/solvent system mixture is heated to 85° C.

Preferably, the protein/solvent system mixture is subjected to a physical stimulus which is heating for a period of about 5 minutes, 10 minutes, 15 minutes, 20 minutes, 25 minutes, 30 minutes, or greater than 30 minutes. The heated protein/solvent system mixture is optionally subjected to subsequent ultrasonication.

The protein solution is then heated such that the liquid solution is held above the sol-gel transition for the protein(s). By modifying the solvent system (for example through selection of the choice of organic acid, the ratio of organic acid to further solvent or through further means) it is possible to modify the sol-gel transition temperature for the protein(s). Through appropriate selection of conditions, it is possible to carefully control the sol-gel transition of the protein thereby controlling the formation of the hydrogel.

Preferably, the protein solution is heated to about or above 70° C. More preferably, the protein is heated to about or above 75° C., about or above 80° C., about or above 85° C. or about 90° C. Even more preferably, the protein is heated to 85° C.

The protein solution may be held at elevated temperature for a time period of about 5 minutes, 10 minutes, 15 minutes, 20 minutes, 25 minutes, 30 minutes, 45 minutes or 1 hour. A preferred time period is at least 30 minutes to enable the proteins to fully solubilise. It is possible to hold the protein solution at an elevated temperature for a longer period of time.

Without wishing to be bound by theory, it is believed that when the plant protein is added to the solvent system the plant protein forms a highly viscous dispersion of insoluble colloidal protein aggregates. Aggregate size may be measured by Dynamic Light Scattering (DLS). Suitable apparatus to measure aggregate size is a Zetasizer Nano S (Malvern).

It is believed that upon heating the protein solution in the presence of a co-solvent system to above the sol-gel transition temperature, the plant proteins partially unfold, resulting in the exposure of hydrophobic amino acids initially buried within the protein native structure. Once partially unfolded, the co-solvents are able to interact with the unfolded protein molecules. For example, an organic acid has greater access to protonate amino acid residues, as well as enabling the formation of anion salt bridges that stabilise hydrophobic interactions. Also, upon heating at elevated temperatures, protein-protein non-covalent intermolecular contacts are disrupted.

Further, it is believed that the application of mechanical agitation, for example ultrasonication, disrupts large colloidal protein aggregates into smaller ones, as well as disrupting protein intermolecular interactions. Using this approach, the size of the protein aggregates can be significantly reduced, before gelation, to particle sizes below 100 nm. Preferably, the method described above comprises protein aggregates with an average size less than 200 nm, preferably less than 150 nm, less than 125 nm, less than 100 nm, less than 90 nm, less than 80 nm, less than 70 nm, less than 60 nm, less than 50 nm, less than 40 nm, or less than 30 nm. It is therefore thought that at this stage in the method the plant-based protein(s) have secondary structures with high levels of α-helical and random coil.

In preferred methods of the present invention, the plant-based protein solution further comprises an additive.

Preferably, said additive is selected from plasticisers, surfactants, rheology modifiers, opacifiers, preservatives, pigments, carbohydrates, gums, polymers, and nanoparticles, or mixtures thereof.

In preferred methods of the present invention, the additive is a plasticiser. Preferably, the additive is selected from glycerol, ethylene glycol, triethylene glycol, tetraethylene glycol, polyethylene glycol, propylene glycol, sorbitol, mannitol, xylitol, fatty acids, glucose, mannose, fructose, sucrose, lactic acid, citric acid, glycolic acid, malic acid, gluconic acid, tartaric acid, ethanolamine, urea, triethanolamine, vegetable oils, lecithin, waxes and amino acids.

In preferred methods of the present invention, the plant-based protein solution further comprises an amine. The presence of an amine in the plant-based protein solution or the external phase in combination with a polyisocyanate in the lipophilic phase allows for the formation of a polymeric coating on the plant-based protein hydrogel.

Preferably, the amine is selected from ethylene diamine, 3,5-diamino-1,2,4-triazole, 1,3-diaminopropane, diethylene triamine, triethylene tetramine, 1,4-diaminobutane, hexamethylene diamine, guanidine or salts thereof, pentaethylene hexamine, diethylenetriamine, bis(3-aminopropyl)amine, and bis(hexamethylene)triamine.

In preferred methods of the present invention, at least 80 wt % of the lipophilic phase, preferably at least 90 wt % of the lipophilic phase, most preferably at least 95 wt % of the lipophilic phase, has at least two Hansen solubility parameters selected from an atomic dispersion force (δD) from 14 to 20, a dipole moment (δP) less than 8, and a hydrogen bonding (δH) from 2.5 to 11.

In preferred methods of the present invention, the lipophilic phase further comprises an oil-miscible solvent.

Preferably, the oil-miscible solvent is a solvent with low volatility (e.g. having a vapour pressure of less than 0.1 Torr at 25° C., preferably less than 0.01 Torr at 25° C., preferably less than 0.001 Torr at 25′C).

Preferably the oil-miscible solvent has low or no odour.

Preferably, the oil-miscible solvent has at least two Hansen solubility parameters selected from: an atomic dispersion force (δD) of less than 20, a dipole moment (δP) of less than 8, and a hydrogen bonding (δH) of less than 11. More preferably, the oil-miscible solvent has at least two Hansen solubility parameters selected from: an atomic dispersion force (δD) of less than 20, a dipole moment (δP) of less than 4, and a hydrogen bonding (δH) of less than 5.

Preferably, the oil-miscible solvent has a density of greater than 1.07 g/cm³. Oil-miscible solvents having this property are advantageously able to prevent creaming of the encapsulates (e.g. in a final product formulation).

Preferably, the oil-miscible solvent contains only low levels of materials with an alcohol functionality (e.g. a primary alcohol functionality). In preferred methods of the present invention, the oil-miscible solvent comprises less than 40% wt alcohol-containing material based upon the total weight of the oil-miscible solvent, more preferably less than 20% wt. In particularly preferred methods of the present invention, the oil-miscible solvent does not comprise an alcohol-containing material.

In preferred methods of the present invention, the oil-miscible solvent in the lipophilic phase is selected from a carboxylic acid ester, a fatty acid ester, a phthalate ester, a triol, a diol, a rosin resin, an isoparaffin, a terpene, and a vegetable oil, or combinations thereof.

Preferably, the oil-miscible solvent in the lipophilic phase is selected from Miglyol® 840, Miglyol® 812 N, Miglyol® 829, Miglyol® 829 ECO, Miglyol® Coco 810, Miglyol® 810 N, Miglyol® 128, Miglyol® 808 Miglyol® T-C7, Miglyol® 8810, Miglyol® PPG 810, Miglyol® OE, Miglyol® DO, and Miglyol® 818, Abalyn®, limonene, benzyl benzoate, diethyl phthalate, isopropyl myristate, triethyl citrate, dipropylene glycol, propylene glycol, triacetin, glycerin, and 1,3 propanediol, or combinations thereof, preferably Miglyol® 812 N. Preferably, the lipophilic phase comprises a material that functions as a viscosity modifier (e.g. Abalyn®).

Preferably, the oil-miscible solvent in the lipophilic phase is a vegetable oil selected from coconut oil, corn oil, canola oil, cottonseed oil, olive oil, palm oil, peanut oil, rapeseed oil, safflower oil, sesame oil, soybean oil, and sunfloweroil. Other examples of vegetable oils are given in the CTFA Cosmetic Ingredient Handbook, J. M. Nikitakis (ed.), 1st ed., The Cosmetic, Toiletry and Fragrance Association, Inc., Washington, 1988. A vegetable oil is an oil that comes from plant sources. Alternatively, the solvent is derived from a vegetable oil.

In preferred methods of the present invention, the lipophilic phase comprises 0 to 80% oil-miscible solvent by volume, preferably 0 to 60%, more preferably 0 to 50%, more preferably 0 to 40%, more preferably 0 to 20%.

In preferred methods of the present invention, the lipophilic phase further comprises a silicon-containing compound. The presence of a silicon-containing compound in the lipophilic phase allows for the formation of a silicon-based coating on the internal surface of the plant-based protein hydrogel (i.e. at the oil droplet interface). Preferably, the silicon-containing compound is selected from tetraethyl orthosilicate, tetrapropyl orthosilicate, dimethyl diethoxysilane and tetramethyl orthosilicate or combinations thereof, preferably tetraethyl orthosilicate.

In preferred methods of the present invention, the lipophilic phase further comprises a polyisocyanate. The presence of a polyisocyanate in the lipophilic phase allows for the formation of a polymeric coating on the plant-based protein hydrogel via reaction between the isocyanate groups and the amine groups present in the protein. Additionally, the presence of a polyisocyanate in the lipophilic phase in combination with an amine in the plant-based protein solution or the external phase allows for the formation of a polymeric coating on the plant-based protein hydrogel via reaction between the isocyanate groups and the amine groups of the added amine. Preferably the polyisocyanate is selected from a trimethylol propane adduct of xylylene diisocyanate (Takenate® D-110N), polyisocyanurate of toluene diisocyanate (Desmodur® RC), or hexamethylele diisocyanate biuret (Desmodur N 100).

In preferred methods of the present invention, the lipophilic phase further comprises a surfactant, preferably an anionic surfactant.

In preferred methods of the present invention, in step (a) the lipophilic phase is cooled to a temperature of less than 20° C. prior to emulsification.

In preferred methods of the present invention, step (a) is conducted at atmospheric pressure. For example, step (a) is a bulk emulsification process (e.g. involving low or high shear mechanical stirring). Thus, in step (a) the primary emulsion is preferably held at a temperature above the sol-gel transition temperature of the plant-based protein(s) for a time period of less than 15 minutes, preferably less than 10 minutes, more preferably less than 5 minutes.

In alternative methods of the present invention, step (a) is conducted at elevated pressure (e.g. in a closed system). For example, step (a) is a membrane emulsification process. Preferably, the membrane emulsification process involves the use of a membrane having a pore size of 3 to 350 μm, preferably 3.5 to 250 μm, more preferably 4 to 150 μm, more preferably 4.5 to 75 μm, more preferably 5 to 50 μm, more preferably 5 to 20 μm, more preferably 5 to 15 μm.

Accordingly, the primary emulsion preferably has a diameter of 3 to 350 μm, preferably 3.5 to 250 μm, more preferably 4 to 150 μm, more preferably 4.5 to 75 μm, more preferably 5 to 50 μm, more preferably 5 to 20 μm, more preferably 5 to 15 μm, as measured by optical microscopy.

In preferred methods of the present invention, the weight ratio of the lipophilic phase to plant-based protein(s) solids in step (a) is in the range 1:5 to 350:1, preferably 1:3 to 60:1, more preferably 1:1 to 40:1, most preferably 2:1 to 25:1.

In preferred methods of the present invention, the external phase comprises a solvent and/or a polymer.

Preferably, the external phase comprises a solvent. More preferably, the solvent is selected from a fatty acid ester, a vegetable oil, a hydrocarbon oil, and silicone oil (i.e. polydimethylsiloxane).

Preferred fatty acid esters are selected from Miglyol® 840, Miglyol® 812 N, Miglyol® 829, Miglyol® 829 ECO, Miglyol® Coco 810, Miglyol® 810 N, Miglyol® 128, Miglyol® 808, Miglyol® T-C7, Miglyol® 8810, Miglyol® PPG 810, Miglyol® OE, Miglyol® DO, and Miglyol® 818, or combinations thereof, preferably Miglyol® 840.

Preferred vegetable oils are selected from coconut oil, corn oil, canola oil, cottonseed oil, olive oil, palm oil, peanut oil, rapeseed oil, safflower oil, sesame oil, soybean oil, and sunflower oil. Other examples of vegetable oils are given in the CTFA Cosmetic Ingredient Handbook, J. M. Nikitakis (ed.), 1st ed., The Cosmetic, Toiletry and Fragrance Association, Inc., Washington, 1988. Alternatively, the solvent is derived from a vegetable oil.

Preferred hydrocarbon oils are paraffin oils.

Preferably, the external phase comprises a polymer. More preferably, the polymer is selected from dextran, pullulan, polyethylene glycol, and polyvinyl alcohol, or combinations thereof, preferably dextran and/or pullulan.

In preferred methods of the present invention, the external phase further comprises a surfactant. Preferably, the surfactant in the external phase is selected from sucrose fatty acid esters such as sucrose stearic acid ester, sucrose palmitic acid ester, sucrose oleic acid ester, sucrose lauric acid ester, sucrose behenic acid ester, and sucrose erucic acid ester; sorbitan fatty acid esters such as sorbitan monostearate, sorbitan tristearate, sorbitan monooleate, sorbitan trioleate, and sorbitan sesquioleate; glyceryl fatty acid esters such as glycerol monostearate and glycerol monooleate; and polyglyceryl fatty acid esters such as diglyceryl tetraisostearate, diglyceryl diisostearate, diglyceryl monoisostearate, and polyglycerol polyricinoleate, preferably polyglycerol polyricinoleate.

In preferred methods of the present invention, the external phase further comprises a silicon-containing compound. The presence of a silicon-containing compound in the external phase allows for the formation of an external silicon-based coating on the plant-based protein hydrogel. Preferably, the silicon-containing compound is selected from tetraethyl orthosilicate, tetrapropyl orthosilicate, dimethyl diethoxysilane and tetramethyl orthosilicate or combinations thereof, preferably tetraethyl orthosilicate.

In preferred methods of the present invention, the external phase further comprises an amine. The presence of an amine in the plant-based protein solution or the external phase in combination with a polyisocyanate in the lipophilic phase allows for the formation of a polymeric coating on the plant-based protein hydrogel via reaction between the isocyanate groups and the amine groups. Preferably, the amine is selected from ethylene diamine, 3,5-diamino-1,2,4-triazole, 1,3-diaminopropane, diethylene triamine, triethylene tetramine, 1,4-diaminobutane, hexamethylene diamine, guanidine or salts thereof, pentaethylene hexamine, diethylenetriamine, bis(3-aminopropyl)amine, and bis(hexamethylene)triamine.

In preferred methods of the present invention, step (b) is conducted at atmospheric pressure. For example, step (b) is a bulk emulsification process (e.g. involving low or high shear mechanical stirring).

In preferred methods of the present invention, step (b) is performed less than 5 min, preferably less than 1 min, more preferably less than 30 seconds, after step (a), In preferred methods of the present invention, in step (b) the external phase is at a temperature that is at least 5° C. higher than the sol-gel transition temperature of the plant-based protein(s), preferably at least 4° C. higher, preferably at least 3° C. higher, preferably at least 2° C. higher, preferably at least 1° C. higher.

In preferred methods of the present invention, the weight ratio of the external phase to the primary emulsion in step (b) is greater than 1:1, more preferably greater than 2:1, more preferably greater than 3:1.

In preferred methods of the present invention, the storage modulus (G′) at 10 rad/s of the plant-based protein hydrogel formed in step (c) is greater than 500 Pa, preferably greater than 1000 Pa, preferably greater than 2500 Pa, preferably greater than 3000 Pa, preferably greater than 4000 Pa.

In preferred methods of the present invention, in step (c) the secondary emulsion is reduced to a temperature below the sol-gel transition temperature of the plant-based protein(s) to form the plant-based protein hydrogel.

Preferably, in step (c) the secondary emulsion is reduced to a temperature of.less than 20° C., preferably less than 10° C., preferably less than 5° C. The secondary emulsion may be held at the reduced temperature for a time period of about 5 minutes, 10 minutes, 15 minutes, 20 minutes, 25 minutes or about 30 minutes.

Without wishing to be bound by theory it is believed that upon cooling the secondary emulsion to below the sol-gel transition temperature, protein-protein non-covalent intermolecular contacts are enabled, thus promoting the self-assembly of plant protein molecules into a network of supramolecular aggregates.

After gelation, the aggregates may be fine stranded. The aggregates may have a median average length of between 50 to 500 nm. The aggregates may have a mean average length of between 50 to 500 nm. 80% of the aggregates may have an average length of between 50 to 500 nm. The aggregates may have a median height of between 5 to 50 nm. The aggregates may have a mean average height of between 5 to 50 nm. 80% of the aggregates may have an average height of between 5 to 50 nm. In a preferred embodiment, the aggregates have a median average length of between 50 to 500 nm and/or a median average height of between 5 to 50 nm.

Without wishing to be bound by theory, it is thought that the method described above allows the plant proteins to aggregate into supramolecular structures held by intermolecular hydrogen bonding interactions, and in particular between the β-strands. Thus, preferably, the plant-based protein hydrogel has high levels of β-sheet intermolecular interactions.

In preferred methods of the present invention, the method does not involve a microfluidic process.

In preferred methods of the present invention, in step (d) the external phase is separated from the microcapsule by decantation, gravity separation, filtration, froth filtration, centrifugation or drying.

In preferred methods of the present invention, in step (d) the external phase is separated from the microcapsule by decantation, gravity separation, filtration, froth filtration, centrifugation or drying prior to washing the microcapsule with water, preferably hard water.

Preferred methods of the present invention further comprise sieving the microcapsule.

Preferred methods of the present invention further comprise subjecting the microcapsule to a post-treatment step. The post-treatment step can advantageously improve the physical and chemical stability of the microcapsules. In particular, the post-treatment step can improve the leakage/diffusion stability of the microcapsules such that they are better able to retain the fragrance material(s) or flavour material(s).

In preferred methods of the present invention, the post-treatment step comprises a non-covalent cross-linking step. Preferably, the non-covalent cross-linking step comprises treating the microcapsule with a non-covalent cross linker selected from sodium tripolyphosphate, sodium hexametaphosphate, and phenolic compounds (e.g. tannic acid, caffeic acid etc.).

In preferred methods of the present invention, the post-treatment step comprises a covalent cross-linking step. Preferably, the covalent cross-linking step comprises treating the microcapsule with a covalent cross linker selected from genipin, epoxy compounds, glyceraldehyde, glutaraldehyde, formaldehyde, glyoxal, dialdehyde starch, microbial transglutaminase, and PolyCup® crosslinking resin, or combinations thereof.

In preferred methods of the present invention, the post-treatment step comprises a coating formation step.

Preferably, the coating formation step comprises treating the microcapsule with a metal compound to form a metal coating. More preferably, the metal compound is selected from a silver compound or a gold compound, preferably a silver compound.

In preferred methods of the present invention, the coating formation step comprises subjecting the microcapsule to a complex coacervation step using a polysaccharide to form a coacervate coating. Preferably, the polysaccharide is selected from xanthan gum, gellan gum, and chitosan or combinations thereof.

In preferred methods of the present invention, the coating formation step comprises treating the microcapsule with an aqueous mineral solution to form a mineral coating. Preferably, the aqueous mineral solution comprises iron salts, calcium salts, phosphate salts, carbonate salts, titanium salts or zinc salts, or combinations thereof.

In preferred methods of the present invention, the coating formation step comprises treating the microcapsule with a polyisocyanate to form a polymeric coating. As outlined above, this can be achieved by the polyisocyanate being present in the lipophilic phase during earlier steps in the method. Preferred polyisocyanates are described above.

In preferred methods of the present invention, the coating formation step comprises treating the microcapsule with a polyisocyanate and an amine to form a polymeric coating. As outlined above, this can be achieved by the polyisocyanate being present in the lipophilic phase and the amine being present in either the plant-based protein solution or the external phase during earlier steps in the method. Preferred polyisocyanates and amines are described above. As will be understood by a skilled person, depending in which phases the polyisocyanate and the amine are present, the polymeric coating can be formed on either the internal surface of the plant-based protein hydrogel (i.e. at the oil droplet interface) or the external surface of the plant-based protein hydrogel (i.e. to form a shell around the microcapsule).

In preferred methods of the present invention, the coating formation step comprises treating the microcapsule with a silicon-containing compound to form a silicon-based coating. As outlined above, this can be achieved by the silicon-containing compound being present in either the lipophilic phase or the external phase during earlier steps in the method. Preferred silicon-containing compounds are described above. As will be understood by a skilled person, depending in which phase the silicon compound is present, the silicon-based coating can be formed on either the internal surface of the plant-based protein hydrogel (i.e. at the oil droplet interface) or the external surface of the plant-based protein hydrogel (i.e. to form a shell around the microcapsule).

As will be understood by a skilled person, it is possible for multiple post-treatment steps to be performed. For example, in a preferred method of the present invention, the microcapsule is subjected to a non-covalent cross-linking step (e.g. using NaTPP) followed by a coating formation step (e.g. using chitosan), and optionally a further non-covalent cross-linking step (e.g. using NaTPP).

Preferred methods of the present invention further comprise drying the microcapsule. The drying may be performed before, after or sequentially with step (d). Preferably, the drying is performed after step (d).

Preferably, the drying is selected from spray drying, fluid bed drying and/or tray drying.

Preferred methods of the present invention further comprise re-suspending the microcapsule in an external phase, preferably an external aqueous phase, preferably hard water or an acidic buffer solution.

The present invention also provides a biodegradable microcapsule obtained by or obtainable by the method as hereinbefore described.

The present invention also provides a method for preparing a biodegradable microcapsule composition, comprising:
(a) emulsifying a lipophilic phase comprising a fragrance or a mixture of fragrances in a plant-based protein solution comprising one or more plant-based protein(s), wherein said lipophilic phase is immiscible with said plant-based protein solution, to give a primary emulsion;
(b) re-emulsifying said primary emulsion in an external phase to give a secondary emulsion, wherein said external phase is immiscible with said primary emulsion; and
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase;
   wherein said plant-based protein solution is at a temperature above the sol-gel transition temperature of the plant-based protein(s) during steps (a) and (b). Preferred features of each of steps (a), (b) and (c) are as described above. Preferred methods of the present invention further comprise:
(d) removing at least part of the external phase from the microcapsule; and
(e) re-suspending the microcapsule in an external phase.

Preferably, the external phase is an external aqueous phase, more preferably hard water or an acidic buffer solution.

Preferred methods of the present invention further comprise adding a suspending agent(s) to said external phase. Preferably, said suspending agent(s) is selected from acacia gum, alginic acid, pectin, xanthan gum, gellan gum, carbomer, dextrin, gelatin, guar gum, hydrogenated vegetable oil category 1, aluminum magnesium silicate, maltodextrin, carboxymethyl cellulose, polymethacrylate, poly vinyl pyrrolidone, sodium alginate, starch, zein, water-insoluble cross-linked polymers such as cross-linked cellulose, cross-linked starch, cross-linked CMC, cross-linked carboxymethyl starch, cross-linked polyacrylate, and cross-linked polyvinylpyrrolidone, and expanded clays such as bentonite and laponite.

The present invention also provides a biodegradable microcapsule composition obtained by or obtainable by the method as hereinbefore described.

The present invention also provides biodegradable microcapsule comprising:
(a) a lipophilic phase comprising at least one fragrance material or flavour material; and
(b) a plant-based protein hydrogel comprising at least one plant-based protein(s);
wherein:
said plant-based protein hydrogel encapsulates said lipophilic phase;
said plant-based protein hydrogel is a covalently-modified or non-covalently modified plant-based protein hydrogel or said plant-based protein hydrogel has a coating deposited thereon; and
   the biodegradation percentage based upon O₂ consumption of the plant-based protein hydrogel as measured according to ISO-14851 version 2019 after 28 days is 60 to 100% based upon the ratio of the Biological Oxygen Demand (BOD) to the Theoretical Oxygen Demand, more preferably 65 to 10%, even more preferably 70 to 100%, even more preferably 75 to 100%, even more preferably 80 to 100%, even more preferably 85 to 100%, most preferably 90 to 100%.

The microcapsules of the present invention are able to effectively encapsulate fragrance materials and/or flavour materials having a range of volatilities, allowing for complex fragrance and flavour profiles to be achieved upon breakage of the microcapsules. The microcapsules comprise a plant-based protein hydrogel, which encapsulates a lipophilic phase comprising at least one fragrance material or flavour material. The plant-based protein hydrogel is a covalently-modified or non-covalently modified plant-based protein hydrogel or has a coating deposited thereon. This reinforcement of the plant-based protein hydrogel means that the fragrance materials and/or flavour materials have a superior retention in the microcapsule. This allows the microcapsules to be particularly useful in a final consumer product.

The microcapsules of the present invention are measured for their biodegradability profile under fresh water conditions for microplastics, as stipulated by ISO-14851 version 2019. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

Accordingly, the microcapsules of the present invention represent environmentally-friendly encapsulation technology, which is particularly suited to use in home care and personal care applications, as if the microcapsules ultimately end up in waterways/the sea they will fully biodegrade in a short amount of time.

In preferred biodegradable microcapsules of the present invention, the biodegradation percentage based upon O₂ consumption of the plant-based protein hydrogel as measured according to ISO-14851 version 2019 after 28 days is 65 to 100% based upon the ratio of the Biological Oxygen Demand (BOD) to the Theoretical Oxygen Demand, more preferably 70 to 100%, even more preferably 75 to 100%, even more preferably 80 to 100%, even more preferably 85 to 100%, most preferably 90 to 100%.

In preferred biodegradable microcapsules of the present invention, the biodegradation percentage based upon O₂ consumption of the microcapsules as measured according to ISO-14851 version 2019 after 28 days is 65 to 100% based upon the ratio of the Biological Oxygen Demand (BOD) to the Theoretical Oxygen Demand, more preferably 70 to 100%, even more preferably 75 to 100%, even more preferably 80 to 100%, even more preferably 85 to 100%, most preferably 90 to 100%.

In preferred biodegradable microcapsules of the present invention, the plant-based protein(s) is obtained from fava bean, mung bean, pea, rice, potato, rapeseed, lentil, chickpea, sunflower seed, pumpkin seed, flax, chia, canola, lupine, alfalfa, moringa, wheat, corn zein or sorghum; preferably the plant protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein, more preferably pea protein and/or potato protein. Such proteins are considered to be low allergenicity proteins.

In preferred biodegradable microcapsules of the present invention, the plant-based protein(s) has been pre-treated with an organic acid. Preferably, the organic acid is acetic acid, lactic acid, formic acid, propionic acid, gluconic acid, an α-hydroxy acid, or a β-hydroxy acid. Particularly preferably, the organic acid is acetic acid or lactic acid.

Preferred α-hydroxy acids include glycolic acid, acetic acid, lactic acid, malic acid, citric acid and tartaric acid, preferably acetic acid and lactic acid. Preferred β-hydroxy acids include β-hydroxypropionic acid, β-hydroxybutyric acid, β-hydroxy β-methylbutyric acid, 2-hydroxybenzoic acid and carnitine.

In preferred biodegradable microcapsules of the present invention, the at least one fragrance material or flavour material is selected from an alcohol, an aldehyde, a ketone, an ester, an ether, an acetate, an alkene, a nitrile, a nitrogenous heterocyclic compound, a sulfurous heterocyclic compound, and a Schiff base. Suitable fragrance materials and flavour materials are described above.

In preferred biodegradable microcapsules of the present invention, the at least one fragrance material or flavour material has a vapour pressure of greater than or equal to 0.0001 Torr at 25° C., preferably greater than or equal to 0.001 Torr at 25° C.

In preferred biodegradable microcapsules of the present invention, the at least one fragrance material or flavour material has a log P greater than or equal to 3.0.

In preferred biodegradable microcapsules of the present invention, the at least one fragrance material or flavour material has at least two Hansen solubility parameters selected from: an atomic dispersion force (δD) from 14 to 20, a dipole moment (δP) of less than 8, and a hydrogen bonding (δH) from 2.5 to 11.

In preferred biodegradable microcapsules of the present invention, the at least one fragrance material or flavour material is part of a fragrance or flavour.

Preferably, the fragrance or flavour contains at least 20 wt % of fragrance material(s) or flavour material(s) with a vapour pressure greater than or equal to 0.001 Torr at 25° C. based upon the total weight of the fragrance or flavour, more preferably 40 wt %, more preferably 60 wt %.

Preferably, the fragrance or flavour contains at least 20 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 20 wt %, more preferably at least 50 wt %, more preferably at least 70 wt %.

Preferably, the fragrance or flavour contains at least 10 wt % of fragrance material(s) or flavour material(s) which have a biodegradation percentage based upon O₂ consumption as measured according to ISO-14851 version 2019 after 28 days of 60 to 100% based upon the ratio of the Biological Oxygen Demand (BOD) to the Theoretical Oxygen Demand, more preferably 65%, even more preferably 70 to 100%, even more preferably 75 to 100%, even more preferably 80 to 100%, even more preferably 85 to 100%, most preferably 90 to 100%. Due to their low aqueous solubility, fragrance or flavours can be added to the biodegradation test on an inert support according to the method in ISO 10634 version 2018: Water quality—Preparation and treatment of poorly water-soluble organic compounds for the subsequent evaluation of their biodegradability in an aqueous medium. In addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (δD) from 14 to 20, a dipole moment (δP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality. In preferred biodegradable microcapsules of the present invention, the fragrance or flavour comprises less than 40% wt alcohol-containing material based upon the total weight of the fragrance or flavour, more preferably less than 20% wt. In particularly preferred biodegradable microcapsules of the present invention, the fragrance or flavour does not comprise an alcohol-containing material.

Preferably, the fragrance or flavour material is of high odour impact. This is advantageous as it ensures that even low levels of fragrance are perceived when released from the microcapsules.

In preferred biodegradable microcapsules of the present invention, at least 80 wt % of the lipophilic phase, preferably at least 90 wt % of the lipophilic phase, most preferably at least 95 wt % of the lipophilic phase, has at least two Hansen solubility parameters selected from an atomic dispersion force (δD) from 14 to 20, a dipole moment (δP) less than 8, and a hydrogen bonding (δH) from 2.5 to 11.

In preferred biodegradable microcapsules of the present invention, said lipophilic phase further comprises an oil-miscible solvent.

Preferably, the oil-miscible solvent is a solvent with low volatility (e.g. having a vapour pressure of less than 0.1 Torr at 25° C., preferably less than 0.01 Torr at 25° C., preferably less than 0.001 Torr at 25° C).

Preferably the oil-miscible solvent has low or no odour.

Preferably, the oil-miscible solvent has at least two Hansen solubility parameters selected from: an atomic dispersion force (δD) of less than 20, a dipole moment (δP) of less than 8, and a hydrogen bonding (δH) of less than 11. More preferably, the oil-miscible solvent has at least two Hansen solubility parameters selected from: an atomic dispersion force (δD) of less than 20, a dipole moment (δP) of less than 4, and a hydrogen bonding (δH) of less than 5.

Preferably, the oil-miscible solvent has a density of greater than 1.07 g/cm³. Oil-miscible solvents having this property are advantageously able to prevent creaming of the encapsulates (e.g. in a final product formulation).

Preferably, the oil-miscible solvent contains only low levels of materials with an alcohol functionality (e.g. a primary alcohol functionality). In preferred biodegradable microcapsules of the present invention, the oil-miscible solvent comprises less than 40% wt alcohol-containing material based upon the total weight of the oil-miscible solvent, more preferably less than 20% wt. In particularly preferred biodegradable microcapsules of the present invention, the oil-miscible solvent does not comprise an alcohol-containing material.

In preferred biodegradable microcapsules of the present invention, the oil-miscible solvent in the lipophilic phase is selected from a carboxylic acid ester, a fatty acid ester, a phthalate ester, a triol, a diol, a rosin resin, an isoparaffin, a terpene, and a vegetable oil, or combinations thereof.

Preferably, the oil-miscible solvent in the lipophilic phase is selected from Miglyol® 840, Miglyol® 812 N, Miglyol® 829, Miglyol® 829 ECO, Miglyol® Coco 810, Miglyol® 810 N, Miglyol® 128, Miglyol® 808, Miglyol® T-C7, Miglyol® 8810, Miglyol® PPG 810 Miglyol® OE, Miglyol® DO, Miglyol® 818, Abalyn®, limonene, benzyl benzoate, diethyl phthalate, isopropyl myristate, triethyl citrate, dipropylene glycol, and propylene glycol, or combinations thereof, preferably Miglyol® 812 N.

Preferably, the oil-miscible solvent in the lipophilic phase is a vegetable oil selected from coconut oil, corn oil, canola oil, cottonseed oil, olive oil, palm oil, peanut oil, rapeseed oil, safflower oil, sesame oil, soybean oil, and sunflower oil. Other examples of vegetable oils are given in the CTFA Cosmetic Ingredient Handbook, J. M. Nikitakis (ed.), 1st ed., The Cosmetic, Toiletry and Fragrance Association, Inc., Washington, 1988. Alternatively, the solvent is derived from a vegetable oil.

In preferred biodegradable microcapsules of the present invention, the lipophilic phase comprises 0 to 80% oil-miscible solvent by volume, preferably 0 to 60%, more preferably 0 to 50%, more preferably 0 to 40%, more preferably 0 to 20%.

In preferred biodegradable microcapsules of the present invention, said lipophilic phase further comprises a surfactant, preferably an anionic surfactant.

In preferred biodegradable microcapsules of the present invention, said plant-based protein hydrogel has been non-covalently modified by a non-covalent cross-linker selected from sodium tripolyphosphate, sodium hexametaphosphate, and phenolic compounds (e.g. tannic acid, caffeic acid etc.).

In preferred biodegradable microcapsules of the present invention, said plant-based protein hydrogel has been covalently modified by a covalent cross-linker selected from genipin, epoxy compounds, glyceraldehyde, glutaraldehyde, formaldehyde, glyoxal, dialdehyde starch, microbial transglutaminase, and PolyCup® crosslinking resin, or combinations thereof.

In preferred biodegradable microcapsules of the present invention, said coating is a metal coating, a silicon-based coating, a polymeric coating, a coacervate coating, or a mineral coating.

In preferred biodegradable microcapsules of the present invention, said coating is a metal coating which is a silver coating or a gold coating, preferably a silver coating.

In preferred biodegradable microcapsules of the present invention, said coating is a silicon-based coating which is formed from a silicon-containing compound. Preferably, said silicon-containing compound is selected from tetraethyl orthosilicate, tetrapropyl orthosilicate, dimethyl diethoxysilane and tetramethyl orthosilicate, or combinations thereof, preferably tetraethyl orthosilicate.

In preferred biodegradable microcapsules of the present invention, said coating is a coacervate coating which is formed from a polysaccharide. Preferably, said polysaccharide is selected from xanthan gum, gellan gum, and chitosan, or combinations thereof.

In preferred biodegradable microcapsules of the present invention, said coating is a mineral coating which is formed from an aqueous mineral solution. Preferably, the aqueous mineral solution comprises iron salts, calcium salts, phosphate salts, carbonate salts, titanium salts or zinc salts, or combinations thereof.

In preferred biodegradable microcapsules of the present invention, said coating is a polymeric coating which is formed from a polyisocyanate, or a polyisocyanate and an amine.

Preferably, said polyisocyanate is selected from a trimethylol propane adduct of xylylene diisocyanate (Takenate® D-110N), polyisocyanurate of toluene diisocyanate (Desmodur® RC), or hexamethylele diisocyanate biuret (Desmodur N 100).

Preferably, said amine is selected from selected from ethylene diamine, 3,5-diamino-1,2,4-triazole, 1,3-diaminopropane, diethylene triamine, triethylene tetramine, 1,4-diaminobutane, hexamethylene diamine, guanidine or salts thereof, pentaethylene hexamine, diethylenetriamine, bis(3-aminopropyl)amine, and bis(hexamethylene)triamine.

Preferred biodegradable microcapsules of the present invention have a coating deposited on the covalently-modified or non-covalently modified plant-based protein hydrogel. Preferred features of the coatings are as described above.

Preferred biodegradable microcapsules of the present invention are core-shell microcapsules.

Preferably, the core-shell microcapsule has a multicore morphology. Alternatively, the core-shell microcapsule has a single core morphology.

Preferred biodegradable microcapsules of the present invention are matrix microcapsules.

Preferred biodegradable microcapsules of the present invention have a diameter of less than or equal to 250 μm, less than or equal to 200 μm, less than or equal to 150 μm, less than or equal to 100 μm, less than or equal to 50 μm, as measured by optical microscopy.

In preferred biodegradable microcapsules of the present invention, said plant-based protein hydrogel comprises plant-based proteins having a protein secondary structure with at least 40% intermolecular R-sheet, at least 50% intermolecular β-sheet, at least 60% intermolecular β-sheet, at least 70% intermolecular β-sheet, at least 80% intermolecular β-sheet, or at least 90% intermolecular β-sheet, wherein the % intermolecular β-sheet content is measured by FTIR (Fourier transform infrared spectroscopy).

In preferred biodegradable microcapsules of the present invention, the at least one fragrance material or flavour material is part of a fragrance or flavour and the microcapsule comprises on a dry basis a weight ratio of fragrance or flavour to plant-based protein(s) in the range 1:5 to 300:1, preferably 1:3 to 50:1, more preferably 1:1 to 25:1, most preferably 2:1 to 20:1.

In preferred biodegradable microcapsules of the present invention, the at least one fragrance material or flavour material is part of a fragrance or flavour and at least 50%, more preferably at least 70%, even more preferably at least 80%, even more preferably at least 90% of the total fragrance or flavour encapsulated is present inside the microcapsule after incubation in hard water at 37° C. for 72 hours, as determined by GC.

In preferred biodegradable microcapsules of the present invention, the at least one fragrance material or flavour material is part of a fragrance or flavour and at least 50%, more preferably at least 70%, even more preferably at least 80%, even more preferably at least 90% of the fragrance or flavour initially encapsulated remains present inside the microcapsule after incubation in hard water at 37° C. for 72 hours, as determined by GC.

In preferred biodegradable microcapsules of the present invention, the at least one fragrance material or flavour material is part of a fragrance or flavour and at least 25%, more preferably at least 40%, even more preferably at least 50%, even more preferably at least 60% of the total fragrance or flavour encapsulated is present inside the microcapsule after incubation in a standard fabric conditioner formulation at 37° C. for 24 hours, as determined by GC.

In preferred biodegradable microcapsules of the present invention, the at least one fragrance material or flavour material is part of a fragrance or flavour and wherein at least 25%, more preferably at least 40%, even more preferably at least 50%, even more preferably at least 60% of the fragrance or flavour initially encapsulated remains present inside the microcapsule after incubation in a standard fabric conditioner formulation at 37° C. for 24 hours, as determined by GC.

In preferred biodegradable microcapsules of the present invention, the microcapsule releases the at least one fragrance material or flavour material when applied and dried onto a surface and upon application of a force to the microcapsule. Such action can result in a sudden and noticeable fragrance or flavour bloom. Preferably, the surface is a bio-surface, glass, paper, card or a textile. Examples of a bio-surface include hair, skin and teeth. Force may be applied directly by touching, rubbing or squeezing or via a device such as brush, comb or mop. Oil being released can be seen visually under a light microscope and the result can also be assessed olfactively.

The rupture-force value (also known as the bursting-force value) of individual microcapsules are measured via a computer-controlled micromanipulation instrument system which possesses lenses and cameras able to image the microcapsules, and which possesses a fine, flat-ended probe connected to a force-transducer (such as the Model 403A available from Aurora Scientific Inc, Canada, or equivalent), as described in: Zhang, Z. et al. (1999), J. Microencapsulation, vol 16, no. 1, pages 117-124, and in: Sun, G. and Zhang, Z. (2001), J. Microencapsulation, vol 18, no. 5, pages 593-602.

In preferred biodegradable microcapsules of the present invention, the microcapsule releases the at least one fragrance material or flavour material as a result of enzymatic degradation of the plant-based protein hydrogel. Preferably, said enzymatic degradation is triggered by enzymes on the skin or in the oral cavity of a human or animal, or by enzymes present in a consumer product (e.g. a cleaning product).

The present invention also provides a composition comprising a biodegradable microcapsule as hereinbefore described and an external phase. The external phase optionally includes a non-encapsulated fragrance material(s) or flavour material(s) that may be the same or different from the encapsulated fragrance material(s) or flavour material(s). The external phase optionally includes other fragrance and flavour delivery systems such as non-biodegradable encapsulates, cyclodextrins or polymer-assisted delivery.

The present invention also provides a consumer product comprising a biodegradable microcapsule as hereinbefore described. Preferably, the consumer product is a fabric conditioner, liquid scent booster, solid scent booster, liquid detergent, powdered detergent, bar soap, shower gel, hair conditioner, shampoo, hair dye or colourant, dishwashing liquid, hard surface cleaner, toilet block, coated fabric, coated paper, wipe, deodorant, antiperspirant, room deodoriser, cosmetic formulation, fine fragrance, perfume additive bead, foodstuff, beverage, toothpaste, mouthwash, chewing gum, nutraceutical product, or pharmaceutical product.

The present invention also provides a method of making a consumer product, comprising:
(a) preparing a biodegradable microcapsule according to the method hereinbefore described; and
(b) mixing said biodegradable microcapsule with a consumer product formulation.

In preferred methods of the present invention, the consumer product is a fabric conditioner, liquid scent booster, solid scent booster, liquid detergent, powdered detergent, bar soap, shower gel, hair conditioner, shampoo, hair dye or colourant, dishwashing liquid, hard surface cleaner, toilet block, coated fabric, coated paper, wipe, deodorant, antiperspirant, room deodoriser, cosmetic formulation, fine fragrance, perfume additive bead, foodstuff, beverage, toothpaste, mouthwash, chewing gum, nutraceutical product, or pharmaceutical product.

In preferred methods of the present invention, the consumer product formulation has a viscosity of less than 100 mPa·s at 20° C. and 50 s⁻¹.

In alternative preferred methods of the present invention, the consumer product formulation has a viscosity of more than 500 mPa·s at 20° C. and 50 s⁻¹, preferably more than 1000 mPa·s at 20° C. and 50 s⁻¹.

The present invention also provides the use of a biodegradable microcapsule as hereinbefore described in a consumer product. Preferably, the consumer product is a fabric conditioner, liquid scent booster, solid scent booster, liquid detergent, powdered detergent, bar soap, shower gel, hair conditioner, shampoo, hair dye or colourant, dishwashing liquid, hard surface cleaner, toilet block, coated fabric, coated paper, wipe, deodorant, antiperspirant, room deodoriser, cosmetic formulation, fine fragrance, perfume additive bead, foodstuff, beverage, toothpaste, mouthwash, chewing gum, nutraceutical product, or pharmaceutical product.

The consumer product described in various aspects of the present invention may take any form suitable for use. Preferably, the consumer product is in the form of a vapour spray, aerosol, emulsion, lotion, liquid, cream, gel, stick, ointment, paste, mousse, powder, granular product, substrate, or semi-solids.

### BRIEF DESCRIPTION OF THE FIGURES

FIG. 1a is an optical microscope image of the microcapsule slurry prepared in Example 1 and diluted 10× in single strength buffer.

FIG. 1b is an optical microscope image of the microcapsule slurry prepared in Example 1, wherein the slurry has been dried onto a glass slide at room temperature for 18 hours and the microcapsules have then been broken by pressing with another glass slide.

FIG. 2a is an optical microscope image of the microcapsule slurry prepared in Example 2 and diluted 10× in single strength buffer.

FIG. 2b is an optical microscope image of the microcapsule slurry prepared in Example 2, wherein the slurry has been dried onto a glass slide at room temperature for 18 hours and the microcapsules have then been broken by pressing with another glass slide.

FIG. 3 is an optical microscope image of the microcapsule slurry prepared in Example 3 and diluted 10× in single strength buffer,

FIG. 4 is an optical microscope image of the microcapsule slurry prepared in Example 4 and diluted 10× in single strength buffer.

FIG. 5 is an optical microscope image of the microcapsule slurry prepared in Example 5 and diluted 10× in single strength buffer.

FIG. 6 is an optical microscope image of the microcapsule slurry prepared in Example 6 and diluted 10× in single strength buffer.

FIGS. 7a and 7b are optical microscope images of the microcapsule slurry prepared in Example 7 at pH 5 and pH 7, respectively, and diluted 10× in single strength buffer.

FIG. 8 is an optical microscope image of the microcapsule slurry prepared in Example 8 and diluted 10× in single strength buffer.

FIG. 9a is an optical microscope image of the microcapsule slurry prepared in Example 9 and diluted 10× in single strength buffer.

FIG. 9b is an optical microscope image of the microcapsule slurry prepared in Example 9, wherein the slurry has been dried onto a glass slide at room temperature for 18 hours and the microcapsules have then been broken by pressing with another glass slide.

FIG. 10 is an optical microscope image of the microcapsule slurry prepared in Example 10 and diluted 10× in single strength buffer.

FIG. 11 is an optical microscope image of the microcapsule slurry prepared in Example 11 and diluted 10× in single strength buffer.

FIG. 12 is an optical microscope image of the microcapsule slurry prepared in Example 12 and diluted 10× in single strength buffer.

FIG. 13 is an optical microscope image of the microcapsule slurry prepared in Example 13 and diluted 10× in single strength buffer.

FIG. 14 is an optical microscope image of the microcapsule slurry prepared in Example 14 and diluted 10× in single strength buffer.

FIG. 15 is an optical microscope image of the microcapsule slurry prepared in Example 15 and diluted 10× in single strength buffer.

FIG. 16 is an optical microscope image of the microcapsule slurry prepared in Example 8 and diluted 10× in single strength buffer.

FIG. 17 is an optical microscope image of the microcapsule slurry prepared in Example 17 and diluted 10× in single strength buffer.

FIG. 18 is an optical microscope image of the microcapsule slurry prepared in Example 18 and diluted 10× in single strength buffer,

FIG. 19 is an 10λ optical microscope image of the washed microcapsule sediment prepared in Example 26.

FIG. 20 is a graph of the evolution of the biodegradation over 28 days of the microcapsule of Example 26 (black squares) and a cellulose control (open squares) measured according to ISO-14851 version 2019.

### EXAMPLES

### Materials

| Material | Supplier | Country |
|---|---|---|
| Acetic acid | Fisher Scientific | United Kingdom |
| Calcium chloride (CaCl₂, dihydrate) | Fisher Scientific | United Kingdom |
| Calcium sulfate (dihydrate) | Fisher Scientific | United Kingdom |
| Chitosan (low molecular weight) | Sigma-Aldrich | United Kingdom |
| Citric acid (monohydrate) | Sigma-Aldrich | United Kingdom |
| ACS reagent, ≥99.0%) | | |
| 1,2-Benzothiazol-3(2H)-one | Fisher Scientific | United Kingdom |
| Gellan gum (high acyl) | Special Ingredients Ltd | United Kingdom |
| Genipin | Fisher Scientific | United Kingdom |
| D(+)-Glucose Anhydrous, Extra Pure, SLR | Fisher Scientific | United Kingdom |
| DL-Glyceraldehyde | Sigma-Aldrich | United Kingdom |
| Hydrochloric acid (HCl) | Sigma-Aldrich | United Kingdom |
| Lactic acid (85 vol %, Food Grade) | Sigma-Aldrich | United Kingdom |
| Microbial transglutaminase Probind TXo | BDF Ingredients | Spain |
| Miglyol ® 812 N | IOI Oleochemical | Germany |
| Miglyol ® 829 | IOI Oleochemical | Germany |
| Myglyol ® 840 | IOI Oleochemical | Germany |
| Pea Protein Isolate (PPI) (80 wt % protein, 4 | Cambridge Commodities | UK |
| wt % carbohydrate) (ProEarth P16109) | Ltd | |
| Polycup ™ LX | Solenis | Spain |
| Polyglycerol polyricinoleate (PGPR) | IOI Oleochemical | Germany |
| (IMWITOR ® 600) | | |
| Potassium hydroxide, ACS reagent, ≥85%, | Sigma-Aldrich | United Kingdom |
| pellets | | |
| Sodium benzoate | Cambridge Commodities | United Kingdom |
| Sodium hexametaphosphate (NaHMP) | Fisher Scientific | United Kingdom |
| Sodium phosphate dibasic anhydrous | Fisher Scientific | United Kingdom |
| Sodium phosphate monobasic anhydrous | Fisher Scientific | United Kingdom |
| Sodium hydroxide | Fisher Scientific | United Kingdom |
| Sodium tripolyphosphate (NaTPP) Alfa | Fisher Scientific | United Kingdom |
| AesarTM | | |
| Tannic acid | Fisher Scientific | United Kingdom |
| Trisodium citrate (dihydrate) | Cambridge Commodities | United Kingdom |
| Tween 80 (Polysorbate 80) | Sigma-Aldrich | United Kingdom |
| Xanthan gum | CP Kelco | United Kingdom |
| Tetraethyl Orthosilicate (TEOS) (98%) | Acros Organics | United Kingdom |
| Sodium Carbonate Anhydrous, Certified AR | Fisher Scientific | United Kingdom |
| for Analysis | | |
| Sodium hydrogen carbonate, Certified AR | Fisher Scientific | United Kingdom |
| for analysis (NaHCO₃) | | |
| Calcium sulfate dihydrate, Puriss., meets | Fisher Scientific | United Kingdom |
| analytical spec. of NF, E 516, 99.0-101.0% | | |
| (based or anhydrous substance) | | |
| (CaSO₄•2H₂O) | | |
| Magnesium sulfate dried (MgSO₄) | Fisher Scientific | United Kingdom |
| Polysorbate 80 (Food Grade) | Sigma-Aldrich | United Kingdom |
| Potassium Chloride, Extra Pure, SLR, Eur. | Fisher Scientific | United Kingdom |
| Ph., Fisher Chemical ™ (KCl) | | |
| Silver nitrate (ACS, >99%) | Fisher Scientific | United Kingdom |
| Stepantex VL-90A | Stepan | USA |
| (1-Hexadecyl)trimethylammonium bromide | Fisher Scientific | United Kingdom |
| Microcrystalline cellulose | Sigma-Aldrich | United Kingdom |

### Fragrance Materials

| Common/trade | | |
|---|---|---|
| name | CAS number | Supplier |
| Delta damascone | 57378-68-4 | Augustus Oils Limited |
| | | and Carvansons |
| Cinnamic alcohol | 104-54-1 | Carvansons |
| Geraniol BJ | 106-24-1 | Carvansons |
| Undecavertol | 81782-77-6 | Carvansons |
| Clonal | 2437-25-4 | Carvansons |
| Dipentene | 138-86-3 | Carvansons |
| Ortho tertiary butyl | 88-41-5 | Carvansons |
| cyclo hexyl acetate | | |
| (OTBCHA) | | |
| Di phenyl oxide | 101-84-8 | Carvansons |
| Benzyl salicylate | 118-58-1 | Carvansons |

### Standard Hard Water and Buffer Compositions

### 1) Hard Water for Washing

This was prepared with the following concentrations of salts in deionised (DI) water:

| | NaHCO₃ | CaSO₄•2H₂O | MgSO₄ | KCl |
|---|---|---|---|---|
| mg/l | 192 | 120 | 120 | 8 |

### 2) Double Strength Buffer for Gellan Gum Dilutions

100 ml of double strength buffer solution with preservative were prepared as follows:
i. 3.808 g of citric acid monohydrate and 0.552 g of trisodium citrate dihydrate were weighed out;
ii. The mixture was completed to 80 g using DI water;
iii. The pH of the solution was adjusted to 3 using 1M HCl and 1M NaOH solutions, and the volume of the solution was completed to 100 g with DI water;
iv. 0.76 g of CaCl₂ dihydrate was added;
v. 0.4 g of sodium benzoate was added;
vi. The solution was placed in an ultrasonic bath at 80° C. for 5-10 minutes (i.e. until complete dissolution was observed);
vii. The solution was allowed to cool down to 20° C.

### 3) Single Strength Buffer for Sample Storage

100 ml of single strength buffer solution with preservative were prepared as follows:
i. 1.904 g of citric acid monohydrate and 0.276 g of trisodium citrate dihydrate were weighed out;
ii. The mixture was completed to 80 g using DI water;
iii. The pH of the solution was adjusted to 3 using 1M HCl and 1M NaOH solutions, and the volume of the solution was completed to 100 g with DI water;
iv, 0.38 g of CaCl₂ dihydrate was added;
v. 0.2 g of sodium benzoate was added;
vi. The solution was placed in an ultrasonic bath at 80° C. for 5-10 min (i.e. until complete dissolution was observed);
vii. The solution was allowed to cool down to 20° C.

### Standard Fabric Conditioner Composition

In some of the following examples, a standard fabric conditioner was employed. This was prepared by mixing the components in Table 1 with an overhead mixer for about 5 minutes at 50° C., then allowing to cool down to 20° C. while mixing.

TABLE 1

| Component | Quantity (wt %) |
|---|---|
| Standard hard water | 87.5 |
| Stepantex VL-90A | 12.2 |
| 25 wt % aqueous calcium chloride | 0.2 |
| 1,2-Benzothiazol-3(2H)-one | 0.1 |

### Measurement Methods

### Microcapsule Fragrance Loading Quantification Via GC

Fragrance levels in the microcapsule can be determined by extraction of the fragrance and injection into a GC column. An Agilent 8890 gas chromatograph is used, equipped with a HP-5MS column, and using hydrogen as carrier gas. Quantification is achieved by use of a calibration curve for the given fragrance material diluted in ethanol.

To measure the total level of fragrance in an encapsulate sample, the capsules must first be broken by sonication, for example 1.0 g of encapsulate in 2.0 g of DI water with a Bandelin Sonopuls HD4200 ultrasonicator, equipped with probe TS104 for 1 minute at 30% amplitude (around 1.5 KJ in total). A few drops of aqueous potassium hydroxide solution (10% wt) are added before sonication to raise the pH of the solution above 11. While sonicating, ice is used to keep the temperature below room temperature (20° C.) to avoid any loss of fragrance through evaporation. A microscope is used to check visually if the capsules are fully broken. If not, sonication is repeated. Ethanol is added to the mixture, which is then mixed and centrifuged. The supernatant is collected and retained as the first extraction. The residue is mixed with ethanol again and centrifuged. The supernatant is collected and added to the first extraction. This is then diluted as appropriate and injected into the GC for analysis.

To measure the level of free fragrance in the sample (i.e. the level of un-encapsulated fragrance), the encapsulate slurry is well mixed in DI water and filtered using a sterile cell strainer (40 μm Nylon mesh). The filtrate is diluted with ethanol and injected into the GC for analysis.

The encapsulated fragrance level is the difference between the total fragrance and the free fragrance levels.

### Microcapsule Fragrance Leakage in Fabric Conditioner Quantification Via GC

Fragrance levels in a fabric conditioner can be determined by extraction of the fragrance and injection into a GC column. An Agilent 8890 gas chromatograph is used, equipped with a HP-5MS column, and using hydrogen as carrier gas. Quantification is achieved by use of a calibration curve for the given fragrance. 3.0 g of fabric conditioner is extracted using 3 ml of n-heptane three times. The extraction is achieved with gentle up-and-down hand shaking (30 times×2) to avoid the formation of an emulsion, and then centrifuged for 10 minutes at 4300 rcf. The n-heptane layers are combined, further diluted with n-heptane as appropriate and injected into the GC for analysis.

In order to determine the level of fragrance leakage in fabric conditioner, the microcapsules are mixed into a fabric conditioner formulation, and 10 g aliquots of this mixture are sealed in glass vials (one aliquot per time point). The level of free and total fragrance is measured in each glass vial after being incubated at 37° C. for a set period of time.

To measure the level of free fragrance in the sample, the fabric conditioner is filtered using a sterile cell strainer (40 μm Nylon mesh). 3.0 g of the filtrate is extracted using 3 ml of n-heptane three times. The extraction is achieved with gentle up-and-down hand shaking (30 times×2) to avoid the formation of an emulsion, and then centrifuged for 10 minutes at 4300 rcf. The n-heptane layers are combined, further diluted with n-heptane as appropriate and injected into the GC for analysis.

In order to measure the total level of fragrance in a fabric conditioner sample, the encapsulate must first be broken by sonication, for example 5 ml of fabric conditioner, with a Bandelin Sonopuls HD4200 ultrasonicator, equipped with probe TS104 for 1 min at 30% amplitude (around 1.5 KJ in total). While sonicating, ice is used to keep the temperature below room temperature to avoid any loss of fragrance through evaporation. A microscope is used to check visually if the capsules are fully broken. If not, sonication is repeated. 3.0 g of the sonicated fabric conditioner sample is then extracted with 3 ml of n-heptane. The extraction is achieved by gentle up-and-down hand shaking (30 times×2) to avoid the formation of an emulsion. The sample is centrifuged for 10 minutes at 4300 rcf. The supernatant is collected and retained as the first extraction. The residue is mixed again with 3 ml of n-heptane and the extraction is repeated twice more. The three extractions are combined, diluted as appropriate and injected into the GC for analysis.

The leakage level is the percentage of the total fragrance that is present as free fragrance at each point in time. The encapsulated fragrance level after storage, for a given amount of time, is the difference between the total fragrance and the free fragrance levels at that point in time.

### Microcapsule Slurry Olfactive Evaluation

For any given encapsulate, the total fragrance level is quantified via GC as described herein. Based on the encapsulate loading the material is diluted to deliver the appropriate level of fragrance for an olfactive assessment. Optionally, preservatives and suspending agents are added to the dilute encapsulate sample.

50 μl of dilute encapsulate is pipetted onto a standard microscope glass slide so that most of the surface area is covered, leaving the edges clear for handling the slide. This is repeated at least four times.

50 μl of dilute encapsulate slurry is also pipetted onto a rectangular fragrance evaluation card blotter (approx. 9 cm×5 cm). This is repeated at least in duplicate.

Slides were evaluated by trained evaluators at different points in time:
1) Less than 30 seconds after application;
2) After being left overnight to dry at 20° C.;
3) After being left overnight to dry at 20° C. and then rubbed with a gloved index finger with 10 strokes (in either direction);
4) After being left overnight to dry at 20° C. and then pressed with another glass slide for 10 seconds and removed.

Blotters were evaluated by trained evaluators at different points in time:
1) Less than 30 seconds after application;
2) After being left overnight to dry at 20° C.;
3) After being left overnight to dry at 20° C. and then rubbed with another clean blotter with 10 strokes (in either direction).

The evaluators graded the strength according to the following scale and provided comments on the character of the fragrance:

| Odour strength | Description |
|---|---|
| 5 | Very strong |
| 4 | Strong |
| 3 | Moderate |
| 2 | Weak |
| 1 | Very weak |
| 0 | No odour |

### Optical Microscopy

Optical microscopy images were obtained using an openFrame microscope equipped with a CellCam 200CR camera, Aura Pro phase contrast illuminator and universal plan fluorite objectives at 4×, 10× and 20×.

### Microcapsule Size Measurement by Optical Microscopy

Particle sizes were taken from the mean size measurements of 50 microcapsules, for each respective sample. The optical microscope was calibrated using the grid of a Hirschmann counting chamber, (Fuchs Rosenthal). Using the straight line tool in ImageJ 1.53, particle diameters were measured from two center-edges with the overlay-text feature enabled to avoid repeating capsules.

### Example 1—Preparation of Delta-Damascone Microcapsules

### a) Preparation of Protein Solution in Water

A protein solution was prepared by suspending 10.0 wt % of Pea Protein Isolate (PPI) in 48.9 wt % of DI water and then adding 41.1 wt % of 85 vol % lactic acid. The mixture was ultrasonicated using a Hielscher UIP1000hdT Ultrasonic Homogenizer, until 0.6-0.7 kJ of energy had been delivered per ml of protein dispersion. The temperature was maintained above 80° C. using a water bath.

### b) Preparation of Lipophilic Phase

The oil phase was prepared by mixing 80% (v/v) of the fragrance material, delta-damascone, and 20% (v/v) of Miglyol® 812 N and cooling to between 0 and 5° C.

### c) Preparation of External Phase

The external phase was prepared by mixing Miglyol® 840 containing 0.5 wt % of PGPR by hand and then placing in a water bath at 53° C. for at least 30 minutes.

### d) Preparation of Primary Emulsion

A Micropore AXF-1 device with a hydrophilic 10 μm membrane and a 9.5 mm insert was used to produce the primary emulsion of fragrance oil in aqueous protein solution. Flow rates of 80 ml/min of oil phase and 120 ml/min of protein solution (ratio of oil to water phases of 2:3) delivered a uniform, continuous and stable O/W (“oil-in-water”) emulsion with droplet size of approximately 40 μm.

Prior to loading with the protein solution, the Micropore system was passivated with 85% lactic acid and pre-heated for 30 minutes. The protein solution was processed as fast as possible, within about 5 minutes, to prevent its gelation whilst in the Micropore device.

### e) Preparation of Secondary Emulsion and Microcapsules

The resultant primary emulsion was poured directly into the external phase, at a ratio of primary emulsion to external phase of 1:4, at a rate of 200 ml/min, into a glass beaker mixed by a Heildolph Hei-TORQUE Core overhead stirrer at 1150 rpm.

The external phase was previously held in a water bath at 53° C., 5° C. higher than the gelation point of the primary O/W emulsion. After the addition of primary emulsion was complete, the secondary emulsion was mixed for 2 minutes at 800 rpm without further temperature control. The beaker contents were then cooled in an ice bath, with stirring at 800 rpm for a further 2 minutes. The stirrer speed was then reduced to 500 rpm until 30 minutes after the start of the secondary emulsification. The temperature of the secondary emulsion was below 20° C.

The formed microcapsules were then allowed to fully settle at 4-5° C. in the fridge for up to 2 hours.

### f) Washing of Microcapsules

The settled microcapsules were decanted from the external phase and resuspended in hard water at 250 rpm to ensure the external phase was thoroughly removed from the surface of the microcapsules. The suspension was added to a separating funnel and left to sediment for 30 minutes to 1 hour. The sedimented microcapsules were collected at the bottom of the separating funnel. Hard water was added to resuspend the microcapsules in a clean separating funnel and again left to sediment. This procedure was repeated two more times.

### g) Microcapsule Classification

The sedimented microcapsules were sieved through a 250 μm sieve while washing with hard water. The sieved suspension was collected and resuspended in hard water. The washing procedure was repeated a second time. The sedimented microcapsules were then directly sieved through a 38 μm sieve while washing with hard water. The microcapsules that remained on the sieve (38 μm<d<250 μm) were collected and resuspended in hard water in a separating funnel. The sedimented microcapsules were then directly sieved through a 75 μm sieve while washing with hard water. The microcapsules that remained on the sieve (75 μm<d<250 μm) were collected and resuspended in hard water in a separating funnel.

### h) Final Microcapsules Dispersion

The microcapsules were strained on a 40 μm cell strainer, made up to a 50 wt % slurry in single strength buffer and stored at 4-5° C.

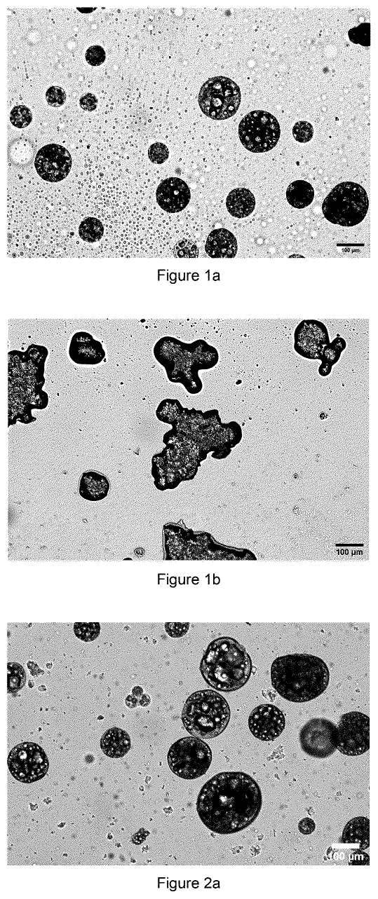

An optical microscope picture of the microcapsule slurry prepared without any post-treatment and diluted 10× in single strength buffer is shown in FIG. 1a. This shows intact spherical multicore microcapsules.

In FIG. 1b the same slurry has been dried onto a glass slide at room temperature for 18 hours and the microcapsules have then been broken by pressing with another glass slide, releasing the encapsulated fragrance oil. This demonstrates the release mechanism so that a fragrance bloom can be noticed from the fragrance encapsulates when they are pressed or rubbed after drying onto a substrate.

The diameter of the microcapsules was measured according to method herein and the average was determined to be 107 μm with a standard deviation of 32 μm.

Fragrance loading analysis was carried out according to the total fragrance loading quantification method described herein. The total delta-damascone loading in the 50 wt % slurry was determined to be 2.1 wt %.

### Example 2—Preparation of Delta-Damascone Microcapsules Modified with NaTPP

Un-crosslinked microcapsules were prepared as in Example 1 up to step 1e,
a) A crosslinking solution of 0.1M NaTPP and 4.0 wt % Polysorbate 80 was prepared.
b) 100 ml of the crosslinking solution was poured over 20 ml of settled microcapsules from Example 1, step e, after having decanted them from the external phase. The suspension was gently stirred by mechanical agitation at 150 rpm at 20° C. for 15 minutes.
c) The cross-linked capsules were then washed and made up to a 50 wt % slurry according to the process in Example 1, steps f to h,

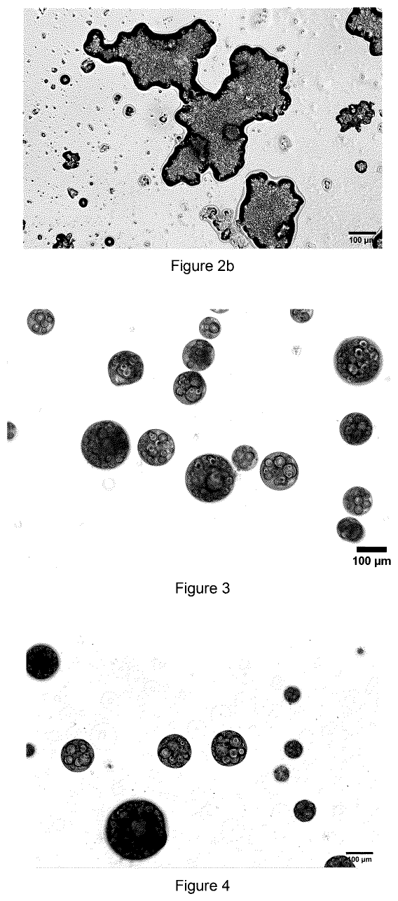

An optical microscope picture of the microcapsule slurry diluted 10× in single strength buffer is shown in FIG. 2a. This shows intact spherical multicore microcapsules which are visibly very similar to those of Example 1, indicating that crosslinking with NaTPP has not reduced the structural integrity of the encapsulates.

In FIG. 2b the same slurry has been dried onto a glass slide at room temperature for 18 hours and the microcapsules have then been broken by pressing with another glass slide, releasing the encapsulated fragrance oil. This demonstrates the release mechanism so that a fragrance bloom can be noticed from the fragrance encapsulates when they are pressed or rubbed after drying onto a substrate.

Fragrance loading analysis was carried out according to the total fragrance loading quantification method described herein. The total delta-damascone loading in the 50 wt % slurry was determined to be 1.6 wt %.

### Example 3—Preparation of Delta-Damascone Microcapsules Modified with Acidic NaTPP

Un-crosslinked microcapsules were prepared as in Example 1 up to step 1g.
a) A crosslinking solution of 0.1M NaTPP and 4.0 wt % Polysorbate 80 was prepared and was adjusted to pH 3 using 1M HCl solution.
b) 100 ml of the crosslinking solution was poured over 20 ml of settled microcapsules after the supernatant was decanted from Example 1, step g. The suspension was gently stirred by mechanical agitation at 100 rpm at room temperature for 15 minutes.
c) The microcapsules were suspended in a separating funnel and sieved through a 250 μm sieve stacked with a 38 μm one to eliminate any possible aggregate resulting from the crosslinking step. The capsules collected on top of the 38 μm sieve were resuspended in a clean separating funnel in hard water to carry out an additional washing step. The decanted capsules were strained through a 40 μm cell strainer for 5 minutes. Any extra moisture was absorbed with a paper tissue. The strained capsules were diluted with single strength buffer to make a 50 wt % slurry.

An optical microscope picture of the microcapsule slurry diluted 10× in single strength buffer is shown in FIG. 3. This shows intact spherical multicore microcapsules which are visibly very similar to those of Example 1, indicating that the crosslinking with NaTPP has not reduced the structural integrity of the encapsulates.

Fragrance loading analysis was carried out according to the total fragrance loading quantification method described herein. The total delta-damascone loading in the 50 wt % slurry was determined to be 0.9 wt %.

### Example 4—Preparation of Delta-Damascone Microcapsules with Layer-by-Layer Deposition of NaTPP-Chitosan-NaTPP

Un-crosslinked microcapsules were prepared as in Example 1 up to step 1g.

2 wt % of chitosan was added to a 2 wt % acetic acid solution and kept under magnetic agitation in a water bath set at 40° C. until complete dissolution was observed. The solution was then stored in the fridge overnight. 2 wt % of NaTPP in a 2 wt % acetic acid solution was also prepared and adjusted to pH 3 using acetic acid.

The next day, 5 g of strained microcapsules were resuspended in 200 ml of hard water and put under mechanical agitation at 150 rpm at room temperature.

21.25 g of the NaTPP solution was added to the microcapsule suspension and left to crosslink for 1.5 hours. Then, 35.41 g of the chitosan solution was added dropwise and left to stir for a further 45 minutes. Finally, another 120.40 g of the NaTPP solution was added and left to crosslink overnight.

The crosslinked capsules were then washed according to the process in Example 3 step c.

An optical microscope picture of the microcapsule slurry diluted 10× in single strength buffer is shown in FIG. 4. This shows intact spherical multicore microcapsules which are visibly very similar to those of Example 1, indicating that the layer-by-layer deposition has not reduced the structural integrity of the encapsulates. In addition, the additional layer is visible for the larger capsules.

Fragrance loading analysis was carried out according to the total fragrance loading quantification method described herein. The total delta-damascone loading in the 50 wt % slurry was determined to be 1.0 wt %. This is lower than the loading of the encapsulates of Example 1 as the encapsulate now includes the additional mass of the deposited layers.

### Example 5—Preparation of Delta-Damascone Microcapsules with Complex Coacervation with Xanthan Gum

Un-crosslinked microcapsules were prepared as in Example 1 up to step 1g.

A 0.1 wt % xanthan gum solution was prepared under magnetic agitation in a water bath set at 50° C. until complete dissolution was observed. The solution was stored in the fridge overnight to allow for the gum to fully hydrate. The next day, the xanthan gum solution was adjusted to pH 3 using 1M HCl.

5 g of strained microcapsules were suspended in 200 ml of hard water and adjusted to pH 3 using 1M HCl. The suspension was put under mechanical agitation at 200 rpm at room temperature. 376 g of xanthan gum solution were slowly added while mixing and left to coacervate for 2 hours.

The crosslinked capsules were then subjected to the washing process as outlined in Example 3 step c.

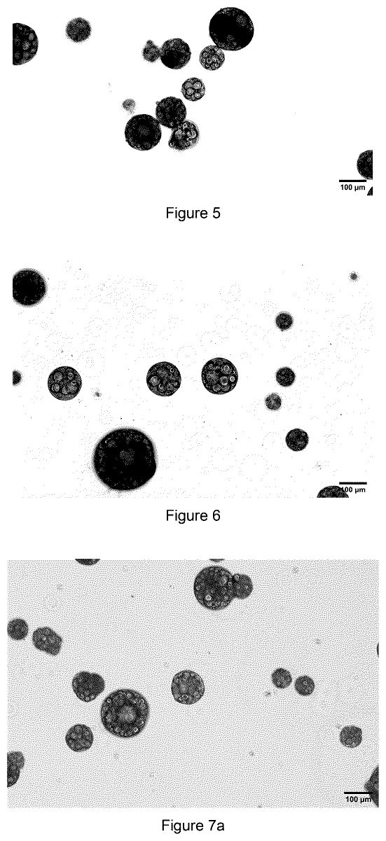

An optical microscope picture of the microcapsule slurry diluted 10× in single strength buffer is shown in FIG. 5. This shows intact spherical multicore microcapsules which are visibly very similar to those of Example 1, indicating that the coacervation has not reduced the structural integrity of the encapsulates. In addition, it is possible to observe some of the coacervate layer on the larger capsules.

Fragrance loading analysis was carried out according to the total fragrance loading quantification method described herein. The total delta-damascone loading in the 50 wt % slurry was determined to be 0.8 wt %, This is lower than the loading of the encapsulates of Example 1 as the encapsulate now includes the additional mass of the coacervate layer.

### Example 6—Preparation of Delta-Damascone Microcapsules with Complex Coacervation with Gellan Gum

Un-crosslinked microcapsules were prepared as in Example 1 up to step 1g.

A 0.1 wt % high acyl gellan gum solution was prepared under magnetic agitation in a water bath set at 95° C. until complete dissolution was observed.

5 g of strained microcapsules were suspended in 200 ml of hard water and placed under mechanical agitation at 200 rpm at 95° C. 414 g of the gellan gum solution were slowly added while mixing and the pH was adjusted to 3 using 1M HCl. Heating was maintained for 30 minutes, then stopped and the suspension was allowed to cool to 20° C. while maintaining agitation for 2 hours in total. The crosslinked capsules were then subjected to the washing process as outlined in Example 3 step c.

An optical microscope picture of the microcapsule slurry diluted 10× in single strength buffer is shown in FIG. 6. This shows intact spherical multicore microcapsules which are visibly very similar to those of Example 1, indicating that the coacervation has not reduced the structural integrity of the encapsulates. In addition, it is possible to observe some of the coacervate layer on the larger capsules.

Fragrance loading analysis was carried out according to the total and free fragrance loading quantification method described herein. The total delta-damascone loading in the 50 wt % slurry was determined to be 1.2 wt %. This is lower than the loading of the encapsulates of Example 1 as the encapsulate now includes the additional mass of the coacervate layer. The free fragrance was very low at 0.003 wt %.

### Example 7—Preparation of Delta-Damascone Microcapsules with NaHMP

Un-crosslinked microcapsules were prepared as in Example 1 up to step 1g.

A 0.8 wt % NaHMP solution was prepared under magnetic agitation at 20° C. The solution was adjusted to the desired pH (5 or 7) using 1M HCl and 1M NaOH solutions.

5 g of strained microcapsules were suspended in 150 ml of the 0.8 wt % NaHMP solution, at either pH 5 or 7. The microcapsule suspension was left stirring at 150 rpm for 2 hours. The crosslinked capsules were then subjected to the washing process outlined in Example 3 step c.

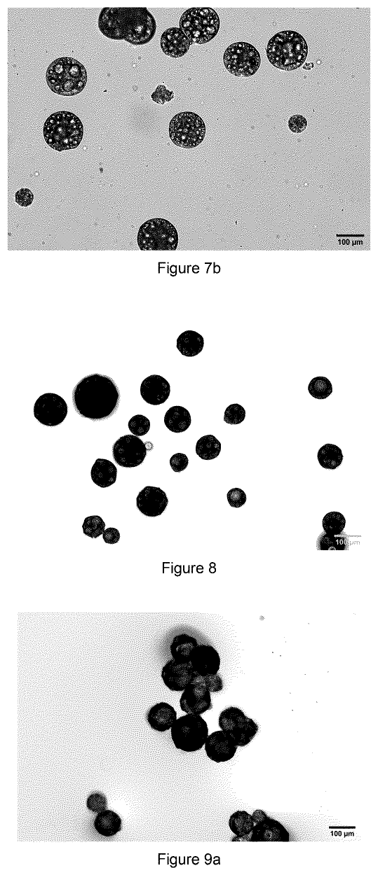

An optical microscope picture of the microcapsule slurry diluted 10× in single strength buffer is shown in FIG. 7a for the slurry at pH 5 and FIG. 7b for the slurry at pH 7. This shows intact spherical multicore microcapsules which are visibly very similar to those of Example 1, indicating that the crosslinking has not reduced the structural integrity of the encapsulates.

Fragrance loading analysis was carried out according to the total fragrance loading quantification method described herein. The total delta-damascone loading in the 50 wt % slurry was determined to be 2.2 and 2.3 wt % for the pH 5 and pH 7 slurries, respectively.

### Example 8—Preparation of Delta-Damascone Microcapsules with Genipin

Un-crosslinked microcapsules were prepared as in Example 1 up to step 1g.

300 mg of genipin were suspended in 10 ml of DI water.

5 g of strained microcapsules were suspended in 290 ml of hard water and the pH was adjusted to 7 using 1M NaOH. The 10 ml of genipin solution were added to the microcapsule suspension under mechanical agitation at 180 rpm at 40° C., and kept stirring for 2 hours. The microcapsules were allowed to crosslink statically for a further 24 hours at 20° C. The crosslinked capsules were then subjected to the washing process as outlined in Example 3 step c.

An optical microscope picture of the microcapsule slurry diluted 10× in single strength buffer is shown in FIG. 8. This shows intact spherical multicore microcapsules which are visibly very similar to those of Example 1 indicating that the crosslinking has not reduced the structural integrity of the encapsulates. The dark colour of the encapsulates is due to the blue pigmentation that confirms that the genipin has covalently bound to the protein shell.

Fragrance loading analysis was carried out according to the total fragrance loading quantification method described herein. The total delta-damascone loading in the 50 wt % slurry was determined to be 0.9 wt %.

### Example 9—Preparation of Delta-Damascone Microcapsules with Tannic Acid

Un-crosslinked microcapsules were prepared as in Example 1 up to step 1g.

A 10 wt % tannic acid solution was prepared by hand shaking and vortexing for a few seconds using a Fisherbrand™ ZX4 IR Vortex Mixer, followed with about 5-10 minutes incubation in an ultrasonic bath set at 50° C.

5 g of strained microcapsules were suspended in 240 ml of hard water and adjusted to pH 3 using 1M HCl. 6 ml of the tannic acid solution was added dropwise to the microcapsule suspension under mechanical agitation at 170 rpm at 50° C., and left stirring for 6 hours. After this time, the suspension was stored in the fridge overnight. The crosslinked capsules were then subjected to the washing process as in Example 3 step c, resulting in brown coloured encapsulates indicating that crosslinking had occurred.

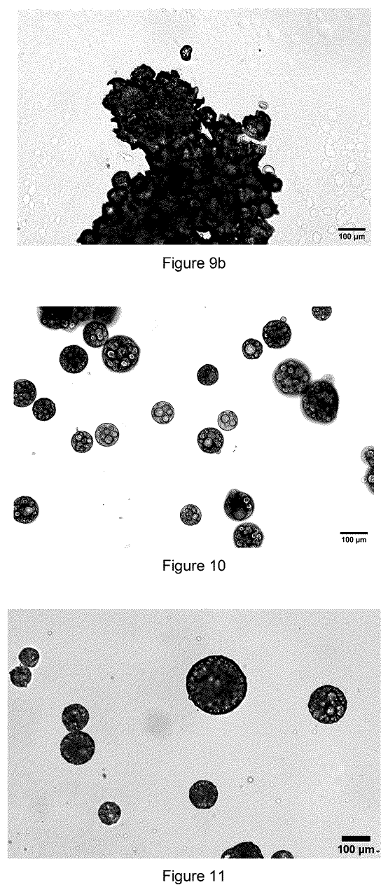

An optical microscope picture of the microcapsule slurry diluted 10× in single strength buffer is shown in FIG. 9a. This shows intact microcapsules which are darker than those of Example 1 and wherein the oil multicores are obscured. Without wishing to be bound by theory, it is thought that the binding of the tannic acid to the plant-based protein renders the external surface of the microcapsule practically opaque.

In FIG. 9b the same slurry has been dried onto a glass slide at room temperature for 18 hours and the microcapsules have then been broken by pressing with another glass slide, releasing the encapsulated fragrance oil. This demonstrates the release mechanism so that a fragrance bloom can be noticed from the fragrance encapsulates when they are pressed or rubbed after drying onto a substrate.

Fragrance loading analysis was carried out according to the total and free fragrance loading quantification method described herein. The total delta-damascone loading in the 50 wt % slurry was determined to be 1.8 wt % and the free fragrance was very low at 0.003 wt %.

### Example 10—Preparation of Delta-Damascone Microcapsules with Glyceraldehyde

Un-crosslinked microcapsules were prepared as in Example 1 up to step 1g.

100 ml of a 0.5 wt % DL-glyceraldehyde solution were prepared and the pH was adjusted to 8 using 1M NaOH.

5 g of strained microcapsules were suspended in the glyceraldehyde solution and put under mechanical agitation at 140 rpm at 20° C. for 24 hours. The crosslinked capsules were then subjected to the washing process outlined in Example 3 step c.

An optical microscope picture of the microcapsule slurry diluted 10× in single strength buffer is shown in FIG. 10. This shows intact spherical multicore microcapsules which are visibly very similar to those of Example 1, indicating that the crosslinking has not reduced the structural integrity of the encapsulates.

Fragrance loading analysis was carried out according to the total and free fragrance loading quantification method described herein. The total delta-damascone loading in the 50 wt % slurry was determined to be 1.2 wt % and the free fragrance was very low at 0.01 wt %.

### Example 11—Preparation of Delta-Damascone Microcapsules with Microbial Transglutaminase

Un-crosslinked microcapsules were prepared as in Example 1 up to step 1g.

A 20 U/ml microbial transglutaminase (m-TG) solution was prepared under magnetic agitation for 10 minutes.

A pH 7 phosphate buffer solution was prepared by dissolving 1.641 g of Na₂HPO₄ and 1.014 g of NaH₂PO₄ in 200 ml of DI water, and adjusting to pH 7 with 1M NaOH.

15 g of strained microcapsules were suspended in 200 ml of phosphate buffer. 1 ml of m-TG solution was added to the suspension under mechanical agitation at 130 rpm in a water bath set at 47° C. and left stirring for 2 hours. The crosslinked capsules were then subjected to the washing process as outlined in Example 3 step c.

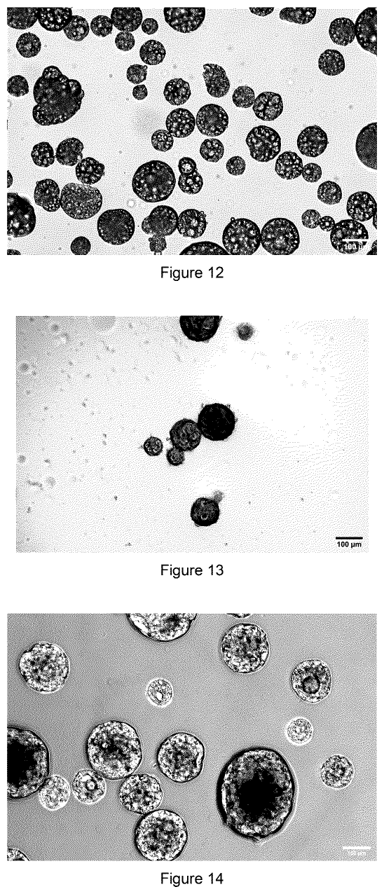

An optical microscope picture of the microcapsule slurry diluted 10× in single strength buffer is shown in FIG. 11. This shows intact spherical multicore microcapsules which are visibly very similar to those of Example 1, indicating that the crosslinking has not reduced the structural integrity of the encapsulates.

Fragrance loading analysis was carried out according to the total fragrance loading quantification method described herein. The total delta-damascone loading in the 50 wt % slurry was determined to be 2.5 wt %.

### Example 12—Preparation of Delta-Damascone Microcapsules with Microbial Transglutaminase and NaHMP

Un-crosslinked microcapsules were prepared as in Example 1 up to step 1g.

A 20 U/ml microbial transglutaminase (m-TG) solution was prepared under magnetic agitation for 10 minutes.

A pH 7 phosphate buffer solution was prepared by dissolving 1.641 g of Na₂HPO₄ and 1.014 g of NaH₂PO₄ in 200 ml of DI water, and adjusting to pH 7 with 1M NaOH.

15 g of strained microcapsules were suspended in 200 ml of phosphate buffer. 1 ml of m-TG solution was added to the suspension under mechanical agitation at 130 rpm in a water bath set at 47° C. and left stirring for 2 hours.

The microcapsules were suspended in a separating funnel and sieved through a 250 μm sieve stacked with a 38 μm one to eliminate any possible aggregate resulting from the crosslinking step. The capsules collected on top of the 38 μm sieve were resuspended in a clean separating funnel in hard water to carry out an additional washing step. The decanted capsules were strained through a 40 μm cell strainer for 5 minutes. Any extra moisture was absorbed with a paper tissue.

A 0.8 wt % NaHMP solution was prepared under magnetic agitation at room temperature. The solution was adjusted to pH 7 using 1M NaOH.

5 g of strained microcapsules were suspended in 150 ml of the NaHMP solution and left stirring at 150 rpm for 2 hours. The cross-linked capsules then went through the washing process as in Example 3 step c.

An optical microscope picture of the microcapsule slurry diluted 10× in single strength buffer is shown in FIG. 12. This shows intact spherical multicore microcapsules which are visibly very similar to those of Example 1 indicating that the crosslinking has not reduced the structural integrity of the encapsulates.

Fragrance loading analysis was carried out according to the total fragrance loading quantification method described herein. The total delta-damascone loading in the 50 wt % slurry was determined to be 2.4 wt %.

### Example 13—Preparation of Delta-Damascone Microcapsules with Polycup™ Crosslinking Resin

Un-crosslinked microcapsules were prepared as in Example 1 up to step 1g.

0.25 g of crosslinking resin was diluted in 10 ml of DI water.

5 g of strained microcapsules were suspended in 90 ml of hard water and the pH was adjusted to 7 using 1M NaOH. The resin solution was added to the suspension under mechanical agitation at 140 rpm at 90° C., and the mixture was left stirring for 30 minutes. The crosslinked capsules were then subjected to the washing process as in Example 3 step c.

An optical microscope picture of the microcapsule slurry diluted 10× in single strength buffer is shown in FIG. 13. This shows intact microcapsules which appear darker than those of Example 1 and wherein the oil multicores are obscured and there is a darker ring around the shell. This indicates that the surface of the encapsulate has been successfully modified by the resin.

Fragrance loading analysis was carried out according to the total fragrance loading quantification method described herein. The total delta-damascone loading in the 50 wt % slurry was determined to be 2.2 wt %.

### Example 14—Preparation of Delta-Damascone “Hand-Made” Microcapsules Coated with Silica

### a) Preparation of Protein Dispersion in Water

A protein dispersion was prepared by measuring 1.4 g of Pea Protein Isolate with 8.25 g of DI water in a 50 ml falcon tube and shaking to mix. To this, 5.75 g of 85% lactic acid was added and the resultant mixture was ultrasonicated using a Bandelin Homogenizer, until 1.0 kJ of energy had been delivered per ml of protein dispersion.

### b) Preparation of Oil Phase

The oil phase was prepared by weighing out 6 g of delta damascone.

### c) Preparation of External Phase:

The external phase was prepared by mixing Miglyol® 840 containing 2.0 wt % of PGPR by hand, drying by standing with anhydrous MgSO₄, filtering with fluted filter paper, and then placing in a water bath at 53° C. for at least 30 minutes.

### d) Preparation of Primary Oil-In-Water Emulsion

The oil phase in b) was poured into the protein dispersion from a), the tube was closed and shaken by hand 3 times to mix.

### e) Preparation of Secondary Emulsion

300 ml of the external phase from c) was added to a 500 ml beaker and the mixture was kept at 53° C.

The primary oil-in-water emulsion was poured into the pre-heated Miglyol® 840 and was stirred for 2 minutes at 1000 rpm. Stirring was continued for a further 5 minutes at 700 rpm. Agitation was reduced to 500 rpm and 15 g of TEOS was added dropwise over 5 minutes. Once addition was completed, stirring was reduced to 300 rpm and left for 4 hours.

f) Crosslinking with NaTPP

The formed microcapsules were then crosslinked with NaTPP as described in Example 2 steps a to c.

An optical microscope picture of the microcapsule slurry diluted 10× in single strength buffer is shown in FIG. 14. This shows intact microcapsules which appear darker than those of Example 1 and wherein the oil multicores are obscured. This indicates that the surface of the encapsulate has been successfully modified by the deposition of silica.

Fragrance loading analysis was carried out according to the total fragrance loading quantification method described herein. The total delta-damascone loading in the 50 wt % slurry was determined to be 0.8 wt %.

### Example 15—Preparation of Delta-Damascone Microcapsules Coated with Silica

A delta damascone primary emulsion was prepared as in Example 1 up to step 1d.

The external phase was prepared as described in Example 14 step c, and maintained at 53° C.

The primary emulsion was poured directly into the external phase, at a ratio of primary emulsion to external phase of 1:4, at a rate of 200 ml/min, into a glass beaker mixed by a Heildolph Hei-TORQUE Core overhead stirrer at 1150 rpm.

After the addition of the primary emulsion was complete, the secondary emulsion was mixed for 5 minutes at 800 rpm without further temperature control. The beaker contents were then cooled in an ice bath and the stirrer speed lowered to 500 rpm. Tetraethyl orthosilicate (50 g) was then added dropwise over 5 minutes. The stirrer speed was lowered to 300 rpm while cooling with ice for 2 h (temperature maintained at <15° C.). The formed microcapsules were then allowed to fully settle in the ice bath for up to 2 hours.

The capsules were then cross-linked as in Example 2 steps a to c.

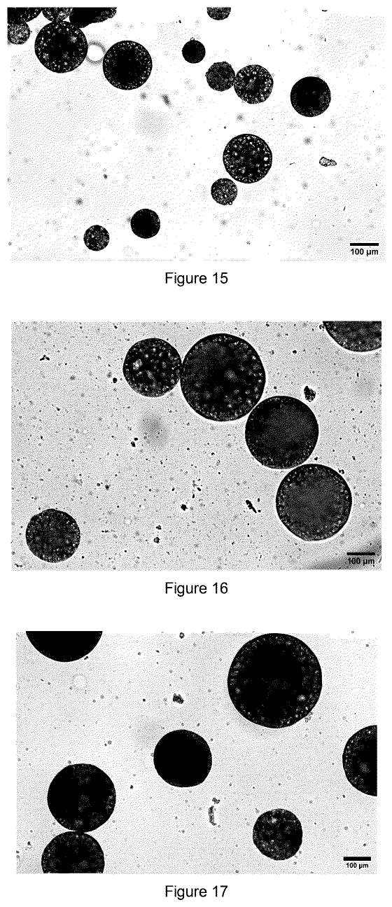

An optical microscope picture of the microcapsule slurry diluted 10× in single strength buffer is shown in FIG. 15. This shows intact microcapsules which appear darker than those of Example 1 and wherein the oil multicores are obscured. This indicates that the surface of the encapsulate has been successfully modified by the deposition of silica.

Fragrance loading analysis was carried out according to the total. fragrance loading quantification method described herein. The total delta-damascone loading in the 50 wt % slurry was determined to be 0.6 wt %.

### Example 16—Preparation of Fragrance Microcapsules Coated with Silver

Crosslinked microcapsules were prepared as in Example 2 and then washed with DI water to remove the single strength buffer.

To 0.73 g of strained capsules was added 8 ml of DI water and 4 ml of equimolar AgNO₃/glucose (200 mM) solution and the mixture left to stir mechanically for 2 minutes at 100 rpm. Then, 8 ml of 0.1M Na₂CO₃ was slowly added to the mixture dropwise and left to stir at 100 rpm for 1 h. Capsules were washed with hard water and made into a 50 wt % slurry in single strength buffer.

An optical microscope picture of the microcapsule slurry diluted 10× in single strength buffer is shown in FIG. 16. This shows intact microcapsules which appear darker than those of Example 1 and wherein the oil multicores are obscured. This indicates that the surface of the encapsulate has been successfully modified by the deposition of silver.

Fragrance loading analysis was carried out according to the total fragrance loading quantification method described herein. The total delta-damascone loading in the 50 wt % slurry was determined to be 1.1 wt %.

### Example 17—Preparation of Fragrance Microcapsules Coated with Silica and Silver

Silica coated microcapsules were prepared as in Example 15 and then washed with DI water to remove the single strength buffer.

To 8 ml of a 50 vol % slurry of silica coated microcapsules was added 4 ml of equimolar AgNO₃/glucose (200 mM) solution and the mixture left to stir mechanically for 2 minutes at 100 rpm. Then, 8 ml of 0.1M Na₂CO₃ was slowly added to the mixture dropwise and left to stir at 100 rpm for 1 h. Capsules were washed with hard water and made into a 50 wt % slurry in single strength buffer.

An optical microscope picture of the microcapsule slurry diluted 10× in single strength buffer is shown in FIG. 17. This shows intact microcapsules which appear even darker than those of Example 15. This indicates that the surface of the encapsulate has been successfully further modified by the deposition of silver.

Fragrance loading analysis was carried out according to the total fragrance loading quantification method described herein. The total delta-damascone loading in the 50 wt % slurry was determined to be 0.6 wt %.

### Example 18—Preparation of Fragrance Microcapsules Containing Internal Silica Layer

A protein solution was prepared as in Example 1 step a. An oil phase containing tetraethyl orthosilicate was prepared by mixing 80% (w/w) of the fragrance material, delta-damascone, 15% (w/w) of Miglyol® 812 N and 5% (w/w) of tetraethyl orthosilicate, and cooling to between 0 and 5° C.

The oil phase was encapsulated as in Example 1 steps c to e, then crosslinked as in Example 2 steps a to c.

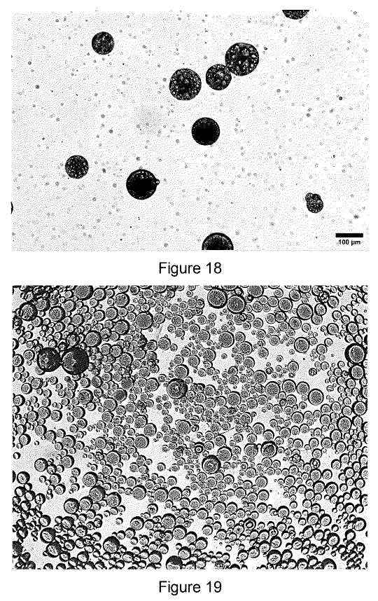

An optical microscope picture of the microcapsule slurry diluted 10× in single strength buffer is shown in FIG. 18. This shows intact microcapsules which appear darker than those of Example 1 and wherein the oil multicores are no longer visible in some cases. This indicates that an internal layer of silica has been successfully formed within the microcapsule.

Fragrance loading analysis was carried out according to the total fragrance loading quantification method described herein. The total delta-damascone loading in the 50 wt % slurry was determined to be 1.1 wt %.

### Example 19—Fragrance Microcapsules

A lipophilic phase of Delta-damascone and Miglyol® 812 N was encapsulated according to the process in Examples 1 and 2 but with variations to the ratio of fragrance material to solvent in the primary emulsion, the amount of sonication energy inputted to the PPI solution and the level of PGPR in the external phase.

In the final washing stage of the process the capsules were filtered down to a specified size range as shown in Table 2 and prepared as a 50 vol % slurry in single strength buffer and analysed for total fragrance loading according to the method herein described.

TABLE 2

| | | Sonication | | Total wt % of | | |
|---|---|---|---|---|---|---|
| Slurry | Fragrance:solvent | energy | PGPR | fragrance in 50 | Particle | |
| Sample | ratio | (kJ/ml) | (wt %) | vol % slurry | size (μm) | Final pH |
| 19A | 80:20 | 1.0 | 0.5 | 2.9 | 75-250 | 3.2 |
| 19B | 80:20 | 1.0 | 1.0 | 1.8 | <250 | 3.1 |
| 19C | 50:50 | 1.0 | 4.0 | 1.4 | 75-250 | 3.2 |
| 19D | 50:50 | 0.6 | 4.0 | 1.0 | 75-250 | 3.2 |

The results show that the process conditions can be optimised to deliver encapsulates with different loading levels for a given fragrance material. For delta-damascone, the highest loading was achieved with the higher sonication energy, the lowest level of PGPR, using a lower level of Miglyol® 812 N in the lipophilic phase and filtering off the finer particles.

### Example 20—Olfactive Performance of Delta-Damascone Microcapsules

A diluted slurry in gellan gum solution was made from sample 19B in Example 19 so that it contained 0.05 wt % of delta-damascone.

The solution of gellan gum was prepared by mixing 19 mg of gellan gum with 30 g of DI water in a 50 ml Falcon tube. The mixture was heated to 95° C. for 20 minutes with stirring. After cooling to room temperature, 30 g of double strength buffer solution were added. Encapsulate slurry was added to the gellan gum solution using a micropipette with a low shear tip, and the sample tube was inverted repeatedly to mix.

The samples were evaluated olfactively according to the method herein described, and the results are shown in Table 3.

TABLE 3

| | | Sample 19B | |
|---|---|---|---|
| Substrate: | | Glass | Card |
| Initial | Strength | 4.5 | 4.5 |
| application | Character | Herbal, green, slightly | Green, herbal, fruity - |
| | | fruity - berry/red | red, slightly woody |
| 24 hours | Strength | 0.5 | 0.5 |
| | Character | Not discernible | Not discernible |
| Rubbing | Strength | 4.0 | 2.75 |
| | Character | Herbal, fruity, slightly green | Fruity -typical damascone, |
| | | | slightly green |
| Pressure | Strength | 4.25 | Not applicable |
| with a | Character | Very herbal, green, | |
| slide | | slightly fruity | |

On initial application there was a strong odour indicating that there was some free fragrance present in the slurry that was either not encapsulated or loosely associated with the outside of the capsule. The initial character was typical of the fragrance material delta-damascone and the slight difference between the substrates was due to the liquid having been absorbed into the card and not the glass slide.

After 24 hours of drying, the free fragrance had mostly evaporated and there was hardly any fragrance odour remaining.

On rubbing both the dried glass and card there was a noticeable release of the fragrance material. On glass, there was a strong odour with the typical herbal fruity note associated with a high level of delta-damascone. On card, there was a moderately strong odour with a more fruity note typical of delta-damascone at a more moderate level. It was believed that the difference in performance on the two substrates was due to all the capsules being available for release on glass whilst the texture of the card meant that some encapsulates may have been hidden within the card's fibrous structure and remained unbroken.

On pressing the dried glass slide with another clean slide there was a very sudden release of fragrance from breaking all the capsules at the same time. This resulted in the strongest release profile and a very herbal and green note due to the high level of delta-damascone released.

### Example 21—Microcapsules Containing Fragrance Accords

Two batches of microcapsules were prepared according to the method in Example 2, encapsulating one of two mixtures of fragrance materials. Table 4 gives the composition of the 2 different fragrance accords which were both equi-weight formulations. Composition 1 comprises only moderately volatile fragrance materials of similar volatility with a range of hydrophobicities from very low to very high. Composition 2 comprises materials of similar hydrophobicity with the full range of volatilies including low, moderate and high volatility materials. Both fragrance compositions had fragrance materials with HSP parameters in the range δD from 15 to 20, δP from 1 to 7 and δH from 2.5 to 13.

TABLE 4

| | Vapour | | | | | | | |
|---|---|---|---|---|---|---|---|---|
| Perfume | pressure | | HSP | | | | Composition | Composition |
| material | (Torr) | LogP | δD | δP | δH | Functionality | 1 (oil wt %) | 2 (oil wt %) |
| Cinnamic | 0.0117 | 1.69 | 19.1 | 6.0 | 13.0 | Primary | 20 | 0 |
| alcohol | | | | | | alcohol | | |
| Geraniol | 0.0133 | 3.42 | 16.9 | 4.2 | 7.6 | Primary | 20 | 0 |
| | | | | | | alcohol | | |
| Undecavertol | 0.0107 | 3.97 | 16.4 | 3.4 | 7.5 | Secondary | 20 | 0 |
| | | | | | | alcohol | | |
| Delta | 0.0102 | 3.56 | 16.9 | 3.5 | 2.6 | Ketone | 20 | 20 |
| damascone | | | | | | | | |
| dodecane | 0.0048 | 5.12 | 16.0 | 5.4 | 3.1 | Nitrile | 20 | 0 |
| nitrile | | | | | | | | |
| Dipentene | 1.5410 | 4.40 | 17.2 | 1.8 | 4.3 | Alkene | 0 | 20 |
| Ortho | 0.1028 | 4.46 | 15.9 | 1.9 | 2.9 | Ester | 0 | 20 |
| tertiary butyl | | | | | | | | |
| cyclo hexyl | | | | | | | | |
| acetate | | | | | | | | |
| (OTBCHA) | | | | | | | | |
| Diphenyl | 0.0223 | 4.03 | 19.4 | 3.4 | 4.0 | Ether | 0 | 20 |
| oxide | | | | | | | | |
| Benzyl | 0.0002 | 4.09 | 19.3 | 6.5 | 7.8 | Ester | 0 | 20 |
| salicylate | | | | | | | | |

Composition 1 was successfully encapsulated into Example 21A and Composition 2 into Example 21B. The oil composition of these microcapsules was analysed using the fragrance loading quantification method via GC described herein. Results are given in Table 5.

TABLE 5

| | Composition 1 | | Composition 2 | |
|---|---|---|---|---|
| | (encapsulate | Composition 1 | (encapsulate | Composition 2 |
| | wt %) | (oil wt %) | wt %) | (oil wt %) |
| Total fragrance | 1.83 | | 2.93 | |
| Cinnamic alcohol | 0.01 | 0.5 | n/a | n/a |
| Geraniol | 0.11 | 6.0 | n/a | n/a |
| Undecavertol | 0.39 | 21.3 | n/a | n/a |
| Delta damascone | 0.54 | 29.5 | 0.53 | 18.1 |
| dodecane nitrile | 0.79 | 43.2 | n/a | n/a |
| Dipentene | n/a | n/a | 0.63 | 21.5 |
| OTBCHA | n/a | n/a | 0.52 | 17.7 |
| Diphenyl oxide | n/a | n/a | 0.59 | 20.1 |
| Benzyl salicylate | n/a | n/a | 0.66 | 22.5 |

Composition 1 demonstrated that the more hydrophobic fragrance materials were preferentially encapsulated, whilst it was still possible to encapsulate relatively hydrophilic fragrance materials within a fragrance accord.

Composition 2 demonstrated that for fragrance materials of similar hydrophobicity it is possible to encapsulate a full range of volatilities from the least volatile to the most volatile.

The 60% higher loading achieved for Composition 2 versus Composition 1 is likely due to the absence of very low log P materials and the absence of alcohols, particularly primary alcohols.

### Example 22—Olfactive Performance of Microcapsules Containing Fragrance Accord

Microencapsulates of Example 21A were evaluated for their olfactive performance as a diluted dispersion suspended in a gellan gum solution (as prepared in Example 20). The microcapsules diameter was measured according to method herein and the average was determined to be 169 μm with a standard deviation of 65 μm.

The volume of microcapsules required was calculated for 0.1 wt % of fragrance oil. The results are described in Table 6.

TABLE 6

| | | Example 21A | Example 21A |
|---|---|---|---|
| | | 0.1% fragrance oil | 0.1% fragrance oil |
| Substrate | | Glass | Card |
| Initial | Strength | 4.5 | 3.5 |
| application | Character | Very fruity, sweet orange | Very fruity, pear, sweet orange |
| | | (candied), metallic, pear | (candied), slightly floral |
| 17 hours | Strength | 0.5 | 2.0 |
| | Character | Not discernible | Fruity, balsamic |
| Rubbing | Strength | 4.5 | 3.25 |
| | Character | Aldehydic floral, very fruity, | Very |
| | | pear | fruity, pear, slightly spicy, slightly |
| | | | metallic |
| Pressure | Strength | 4.0 | Not applicable |
| with a | Character | Aldehydic floral, very fruity, | |
| slide | | pear | |

On initial application to glass and card, there was a strong odour indicating that there was some free fragrance present in the slurry that was either not encapsulated or loosely associated with the outside of the microcapsules. The initial character was very fruity and sweet. The difference in strength and fragrance character between the substrates was due to the liquid having been absorbed into the card and not the glass slide.

After 17 hours of drying, the free fragrance had mostly evaporated and there was hardly any fragrance odour remaining on the glass. On the card there was a weak fruity odour as the free fragrance was trapped in the card and evaporated more slowly.

On rubbing, both the dried glass and card there was a noticeable release of the fragrance material. On glass, there was a strong odour with an aldehydic floral and very fruity note. On card, there was a moderately strong odour with a more fruity note indicating that fewer encapsulates were broken by rubbing. It was believed that the difference in performance of the two substrates was due to all the capsules being available for release on glass whilst the fibrous texture of the card meant that some capsules may have been hidden within the card and not all were broken on rubbing.

On pressing the dried glass slide with another clean slide there was a very sudden release of fragrance from breaking the capsules, resulting in the strong release profile and an aldehydic floral and fruity note.

### Example 23—Stability of Fragrance Microcapsules in Standard Hard Water

The 50 wt % slurry of the fragrance microcapsules of Examples 1, 2 and 9 was diluted in standard hard water so that the level of fragrance was around 0.1 wt % and the pH was 3.5-4.5. The samples were placed in an incubator at 37° C. and the free and total fragrance was measured initially, after 4 hours, 72 hours and 2 weeks. The fragrance leakage was calculated as free fragrance as a percentage of total fragrance loading for each encapsulate. The results are shown in Table 7.

TABLE 7

| | | % fragrance | % fragrance | % fragrance |
|---|---|---|---|---|
| | | leakage after | leakage after | leakage after |
| | | 4 hours in | 72 hours in | 2 weeks in |
| Sample | Post-treatment | hard water | hard water | hard water |
| Example 1 | None | 5.8 | 3.4 | 5.7 |
| Example 2 | NaTPP | 4.3 | 4.8 | 6.0 |
| Example 9 | Tannic acid | 4.3 | 7.3 | 12.7 |

The proportion of the fragrance that was free remained low for up to 2 weeks indicating that both the un-crosslinked sample and the post-treated samples were stable in standard hard water.

### Example 24—Stability of Fragrance Microcapsules in Fabric Conditioner

Fragrance microcapsules were added to a simple unperfumed fabric conditioner formulation to test the stability of the microcapsules in this very challenging environment, which is believed to be due to the combination of a surfactant and a low viscosity product with high water availability.

The fabric conditioner was prepared as described above. For each microcapsule to be tested, the amount added to the unperfumed fabric conditioner was calculated according to its loading so that the level of fragrance was around 0.1 wt % for each stability test.

Fabric conditioner samples containing the microcapsules of Examples 1, 6, 9, 15, 16, 17 and 18 were placed in an incubator at 37° C. and the free and total fragrance in the fabric conditioner was measured via the methods herein described less than 30 seconds after mixing with the fabric conditioner and after 4 and 24 hours in the incubator. The fragrance leakage was calculated as free fragrance as a percentage of total fragrance loading for each encapsulate. The initial level of free fragrance in the slurry for these encapsulates was negligible and could be ignored in this stability test. The results are shown in Table 8.

TABLE 8

| | | % fragrance leakage | | |
|---|---|---|---|---|
| | | less than 30 seconds | % fragrance leakage | % fragrance leakage |
| | | after addition to fabric | after 4 hours in fabric | 24 hours in fabric |
| Sample | Post-treatment | conditioner | conditioner | conditioner |
| Example 1 | None | 28.8 | 88.4 | 90.0 |
| Example 9 | Tannic acid | 11.3 | 43.4 | 57.2 |
| Example 6 | Gellan gum | 17.3 | 77.6 | 65.3 |
| Example | Silica exterior | 25.2 | 38.8 | 60.5 |
| 15 | | | | |
| Example | Silver coating | 18.1 | 72.9 | N/A |
| 16 | | | | |
| Example | Silica + silver | 17.8 | 40.8 | 97.7 |
| 17 | exterior | | | |
| Example | Silica interior | 11.7 | 48.5 | 81.4 |
| 18 | | | | |

The results show that the un-crosslinked microcapsules (Example 1) started to leak fragrance as soon as they were mixed into the fabric conditioner, and after 4 hours they had lost 88% of the encapsulated fragrance. The various post-treatments reduced the leakage from the microcapsules less than 30 seconds after mixing and to between 39 and 78% after 4 hours. All the post treatments provided some initial protection from leakage in fabric conditioner demonstrating that the encapsulates could be dosed into a fabric conditioner for immediate usage either manually or automatically within a washing machine. The best performing post-treatments were tannic acid cross-linking, silica coating and the combination of silica and silver coating. At 24 hours, the best performing post-treatments were tannic acid cross-linking and an exterior silica coating, demonstrating these post-treated encapsulates are more resistant to leakage and have the potential to be used in a fabric conditioner application to deliver a scent bloom.

### Example 25: Formation of Interfacial Polymerisation Shell

An optional thin polymeric shell may be formed as part of the encapsulate wall in order to provide the plant-based protein microcapsule with an additional barrier to prevent diffusion of the lipophilic components out of the encapsulate and into the external environment. As the structural integrity of the encapsulate is provided by the plant-based protein, this polymeric shell can be very thin. The polymeric shell is created by the in situ reaction between polyisocyanate and diamine. Two possible processing routes to achieve this are discussed below.

1) Interfacial Polymerisation Initiated after the Secondary Emulsification

A first crosslinking agent, such as polyisocyanate, is introduced into the lipophilic phase at a level of 1-20 wt % of said phase before forming the primary emulsion.

The microcapsules are formed following the double emulsification process as described herein, optionally followed by a covalent crosslinking step.

The microcapsule dispersion can be adjusted to pH 7 using either HCl or NaOH solutions. A 3 wt % aqueous solution (pH 7) of the second crosslinking agent, such as diamine, is added at 20° C. to the microcapsule dispersion in an equimolar proportion compared to the polyisocyanate, and at a rate of 1 ml/min, using a syringe pump, to induce polymerisation. The mixture is then kept stirring for 2 hours at 20° C.

The capsules are then left to sediment in the external oil at 4-5° C. and optionally further processed (e.g. washed and crosslinked according to the processes described herein).

2) Interfacial Polymerisation Initiated after Primary Emulsification/Before the Secondary Emulsification

A first crosslinking agent, such as polyisocyanate, is introduced into the lipophilic phase at a level of 1-20 wt % of said phase before forming the primary emulsion.

A diamine is added to the PPI solution in an equimolar proportion compared to the amount of polyisocyanate after having fully dissolved the protein in the lactic acid aqueous solution.

The primary emulsion is then formed as per the process described herein.

The process for double emulsion formation is modified in that the external phase is maintained in a water bath heated up to 90° C. After forming the double emulsion, it is stirred for 10 minutes at 90° C. to allow for curing, and then allowed to cool down to 20° C. It is then further cooled down to 15-10° C., using ice, over 1 hour. Alternatively, the mixture is cooled down from 90 to 10° C. at a controlled rate of 0.2-0.3° C./min.

The capsules are then left to sediment in the external oil at 4-5° C. and optionally further processed (e.g. washed and crosslinked according to the processes described herein).

### Example 26: Biodegradation Testing

Empty microcapsules were prepared as follows for biodegradation testing:

### a) Preparation of Protein Aqueous Organic Acid Solution

Approximately 200 mls of protein solution was prepared by suspending 25 g of Pea Protein Isolate (PPI) in 117.5 g of 0.1M calcium chloride solution and then adding 99 g of 85 vol % lactic acid solution. The mixture was ultrasonicated using a QSonica Q700 Ultrasonic Homogeniser until 0.5 kJ of energy had been delivered per ml of protein solution. This also resulted in heating the solution to around 80° C.

### b) Preparation of External Phase

The external phase was prepared by mixing 500 ml Miglyol® 840 containing 2 wt % of PGPR and then heated in a water bath at 70° C. The oil was stirred at 1000 rpm with a 3-blade impeller in a 1 litre plastic beaker.

### c) Preparation of Secondary Emulsion and Microcapsules

The external phase temperature was kept above the gelation temperature of the acid treated protein solution. The protein solution was poured directly into the external phase and mixed at 1000 rpm with a 3-blade impeller for 1 min whilst being still in the hot water bath to maintain the temperature. The beaker contents were then cooled in an ice bath, with stirring at 500 rpm for a further 15 minutes. The temperature dropped to below 20° C.

The formed microcapsules were then allowed to fully settle at 4-5° C. in the fridge.

### d) Washing of Microcapsules

After 3 days the settled microcapsules were removed from the fridge and decanted from the external phase and resuspended in 0.1M calcium chloride solution with 1.5 wt % polysorbate 80 added. This was stirred at 500 rpm with an overhead stirrer to ensure the external phase was thoroughly removed from the surface of the microcapsules. The suspension was added to a separating funnel and left to sediment for 1 to 2 hours. The sedimented microcapsules were collected at the bottom of the separating funnel. 0.1M trisodium citrate solution was added to resuspend the microcapsules in a clean separating funnel and again left to sediment. The microcapsules formed are shown in FIG. 19.

The sedimented microcapsules were prepared for biodegradation testing by drying in an oven at 85° C. for 1 hr and 30 mins. The dried microcapsules were ground to a powder with an average particle size of 55 microns measured by microscopy.

The microcapsules were tested for their aqueous aerobic biodegradation based upon O₂ consumption after 28 days as measured according to ISO-14851 version 2019. A control material of microcrystalline cellulose was also tested.

The source of the inoculum was a mixture of activated sludge, obtained from different wastewater treatment plants treating domestic and/or industrial sewage. The different sludges were sieved over an 80 μm sieve and mixed in equal parts. The supernatant liquid was removed and replacement with standard mineral medium and the inoculum was aerated for 1 hour.

At the start of the test, each reactor was filled with the same amount of mineral medium. 2 wt % inoculum was added to each reactor to obtain a test medium with a concentration of 21 mg suspended solids/I. The reference and test item were then added directly to their respective reactors. A magnetic rod kept the materials and the growing biomass suspended throughout the test. The reactors were incubated at 21° C.±1° C. in the dark for 28 days. The Biological Oxygen Demand (BOD) was analysed at regular intervals. The percentage of biodegradation based on 02 consumption is expressed as the ratio of the BOD (corrected for the control) to the Theoretical Oxygen Demand (ThOD) of the test material.

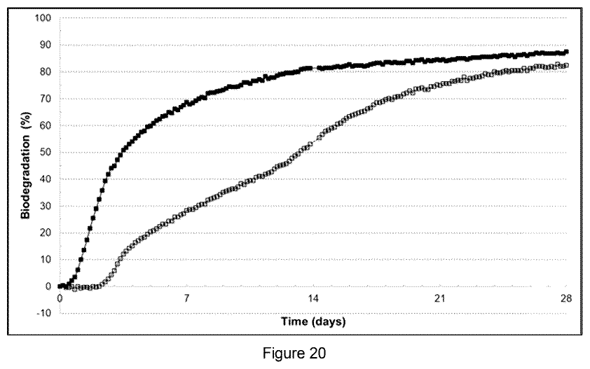

The % biodegradation of the cellulose control was 82.4% after 28 days which means the test is valid as specified by the ISO-14851 method. The % biodegradation of the empty microcapsules was 87.5% after 28 days. The evolution of the biodegradation can be seen in FIG. 20.

1. A method for preparing a biodegradable microcapsule, comprising:
(a) emulsifying a lipophilic phase comprising at least one fragrance material or flavour material in a plant-based protein solution comprising one or more plant-based protein(s) to give a primary emulsion, wherein said lipophilic phase is immiscible with said plant-based protein solution;
(b) re-emulsifying said primary emulsion in an external phase to give a secondary emulsion, wherein said external phase is immiscible with said primary emulsion;
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase; and
(d) separating the external phase from the microcapsule;
wherein said plant-based protein solution is at a temperature above the sol-gel transition temperature of the plant-based protein(s) during steps (a) and (b), and wherein in step (c) the secondary emulsion is reduced to a temperature below the sol-gel transition temperature of the plant-based protein(s).

2. A method according to claim 1, wherein the plant-based protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein.

3. A method according to claim 1, wherein;
the at least one fragrance material or flavour material has a vapour pressure of greater than or equal to 0.0001 Torr at 25° C.; and/or
the at least one fragrance material or flavour material has a log P greater than or equal to 3.0.

4. (canceled)

5. A method according to claim 4, wherein;
the at least one fragrance material or flavour material is part of a fragrance or flavour; and
(i) the fragrance or flavour contains at least 20 wt % of fragrance material(s) or flavour material(s) with a vapour pressure greater than or equal to 0.001 Torr at 25° C. based upon the total weight of the fragrance or flavour; and/or
(ii) the fragrance or flavour contains at least 20 wt % of fragrance material(s) or flavour material(s) with a log P greater than 3.0 based upon the total weight of the fragrance or flavour; and/or
(iii) the fragrance or flavour contains at least 10 wt % of fragrance material(s) or flavour material(s) of natural origin based upon the total weight of the fragrance or flavour and/or
(iv) the fragrance or flavour contains at least 10 wt % of fragrance material(s) or flavour material(s) which have a biodegradation percentage based upon O2 consumption as measured according to ISO-14851 version 2019 after 28 days of 60 to 100% based upon the ratio of the Biological Oxygen Demand (BOD) to the Theoretical Oxygen Demand.

6. A method according to claim 1, wherein the plant-based protein solution comprises one or more plant-based protein(s) in a solvent system, wherein the solvent system comprises miscible co-solvents; wherein a first co-solvent increases solubility of the plant-based protein(s), and a second co-solvent decreases solubility of the plant-based protein(s).

7. A method according to claim 6, wherein the first co-solvent is an organic acid selected from the group consisting of acetic acid, lactic acid, formic acid, gluconic acid, propionic acid, an α-hydroxy acid, or a β-hydroxy acid.

8. A method according to claim 6, wherein the second co-solvent(s) is selected from water, ethanol, and/or ethyl acetate.

9. A method according to claim 1, wherein the plant-based protein solution further comprises an amine, preferably selected from the group consisting of ethylene diamine, 3,5-diamino-1,2,4-triazole, 1,3-diaminopropane, diethylene triamine, triethylene tetramine, 1,4-diaminobutane, hexamethylene diamine, guanidine or salts thereof, pentaethylene hexamine, diethylenetriamine, bis(3-aminopropyl)amine, and bis(hexamethylene)triamine.

10. A method according to claim 1, wherein the lipophilic phase further comprises an oil-miscible solvent, selected from the group consisting of a carboxylic acid ester, a fatty acid ester, a phthalate ester, a triol, a diol, a rosin resin, an isoparaffin, a terpene, and a vegetable oil, and combinations thereof.

11. A method according to claim 1, wherein the lipophilic phase further comprises a polyisocyanate, preferably selected from the group consisting of a trimethylol propane adduct of xylylene diisocyanate (Takenate® D-110N), polyisocyanurate of toluene diisocyanate (Desmodur® RC), or hexamethylele diisocyanate biuret (Desmodur N 100).

12. A method according to claim 1, wherein the lipophilic phase further comprises a silicon-containing compound, preferably selected from the group consisting of tetraethyl orthosilicate, tetrapropyl orthosilicate, dimethyl diethoxysilane and tetramethyl orthosilicate and combinations thereof.

13. A method according to claim 1, wherein in step (a) the lipophilic phase is cooled to a temperature of less than 20° C. prior to emulsification.

14. A method according to claim 1, wherein in step (a) the primary emulsion is held at a temperature above the sol-gel transition temperature of the plant-based protein(s) for a time period of less than 15 minutes.

15. A method according to claim 1, wherein step (a) is a membrane emulsification process.

16. A method according to claim 1, wherein the external phase comprises a solvent and/or a polymer.

17. A method according to claim 1, wherein the external phase further comprises a silicon-containing compound, preferably selected from the group consisting of tetraethyl orthosilicate, tetrapropyl orthosilicate, dimethyl diethoxysilane and tetramethyl orthosilicate and combinations thereof.

18. A method according to claim 1, wherein the external phase further comprises an amine, preferably selected from the group consisting of ethylene diamine, 3,5-diamino-1,2,4-triazole, 1,3-diaminopropane, diethylene triamine, triethylene tetramine, 1,4-diaminobutane, hexamethylene diamine, guanidine or salts thereof, pentaethylene hexamine, diethylenetriamine, bis(3-aminopropyl)amine, and bis(hexamethylene)triamine.

19. A method according to claim 1, wherein step (b) is performed less than 5 min after step (a).

20. A method according to claim 1, wherein in step (b) the external phase is at a temperature that is at least 5° C. higher than the sol-gel transition temperature of the plant-based protein(s).

21. A method according to claim 1, further comprising subjecting the microcapsule to a post-treatment step, preferably wherein the post-treatment step comprises a non-covalent cross-linking step, a covalent cross-linking step or a coating formation step.

22. A method according to claim 21, wherein the non-covalent cross-linking step comprises treating the microcapsule with a non-covalent cross linker selected from sodium tripolyphosphate, sodium hexametaphosphate, and phenolic compounds.

23. A method according to claim 21, wherein the covalent cross-linking step comprises treating the microcapsule with a covalent cross linker selected from genipin, epoxy compounds, glyceraldehyde, glutaraldehyde, formaldehyde, glyoxal, dialdehyde starch, microbial transglutaminase, and PolyCup® crosslinking resin, or combinations thereof.

24. A method according to claim 21, wherein the coating formation step comprises:
(i) treating the microcapsule with a metal compound; and/or
(ii) subjecting the microcapsule to a complex coacervation step using a polysaccharide; and/or
(iii) treating the microcapsule with an aqueous mineral solution.

25. A method for preparing a biodegradable microcapsule composition, comprising:
(a) emulsifying a lipophilic phase comprising a fragrance or a mixture of fragrances in a plant-based protein solution comprising one or more plant-based protein(s), wherein said lipophilic phase is immiscible with said plant-based protein solution, to give a primary emulsion;
(b) re-emulsifying said primary emulsion in an external phase to give a secondary emulsion, wherein said external phase is immiscible with said primary emulsion; and
(c) inducing the plant-based protein(s) to undergo a sol-gel transition to from a plant-based protein hydrogel, wherein said plant-based protein hydrogel encapsulates said lipophilic phase to form a biodegradable microcapsule which is suspended in said external phase;
wherein said plant-based protein solution is at a temperature above the sol-gel transition temperature of the plant-based protein(s) during steps (a) and (b), wherein in step (c) the secondary emulsion is reduced to a temperature below the sol-gel transition temperature of the plant-based protein(s).

26. A biodegradable microcapsule composition obtained by or obtainable by the method of claim 25.

27. A biodegradable microcapsule comprising:
(a) a lipophilic phase comprising at least one fragrance material or flavour material; and
(b) a plant-based protein hydrogel comprising at least one plant-based protein(s); wherein:
said plant-based protein hydrogel encapsulates said lipophilic phase;
said plant-based protein hydrogel is a covalently-modified or non-covalently modified plant-based protein hydrogel or said plant-based protein hydrogel has a coating deposited thereon; and
the biodegradation percentage based upon 02 consumption of the plant-based protein hydrogel as measured according to ISO-14851 version 2019 after 28 days is 60 to 100% based on the ratio of the Biological Oxygen Demand (BOD) to the Theoretical Oxygen Demand.

28. A biodegradable microcapsule according to claim 27, wherein the plant-based protein(s) is selected from the group consisting of pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein.

29. A biodegradable microcapsule according to claim 27, wherein the at least one fragrance material or flavour material has a vapour pressure of greater than or equal to 0.0001 Torr at 25° C.; and/or
the at least one fragrance material or flavour material has a log P greater than or equal to 3.0.

30. A biodegradable microcapsule according to claim 27, wherein the at least one fragrance material or flavour material is part of a fragrance or flavour.

31. A biodegradable microcapsule according to claim 30, wherein;
(i) the fragrance or flavour contains at least 20 wt % of fragrance material(s) or flavour material(s) with a vapour pressure greater than or equal to 0.001 Torr at 25° C. based upon the total weight of the fragrance or flavour; and/or
(ii) the fragrance or flavour contains at least 20 wt % of fragrance material(s) or flavour material(s) with a log P greater than 3.0; and/or
(iii) the fragrance or flavour contains at least 10 wt % of fragrance material(s) or flavour material(s) of natural origin based upon the total weight of the fragrance or flavour and/or
(iv) the fragrance or flavour contains at least 10 wt % of fragrance material(s) or flavour material(s) which have a biodegradation percentage based upon 02 consumption as measured according to ISO-14851 version 2019 after 28 days of 60 to 100% based upon the ratio of the Biological Oxygen Demand (BOD) to the Theoretical Oxygen Demand.

32. A biodegradable microcapsule according to claim 27, wherein said lipophilic phase further comprises an oil-miscible solvent, preferably selected from the group consisting of a carboxylic acid ester, a phthalate ester, a fatty acid ester, a triol, a diol, a rosin resin, an isoparaffin, a terpene, and a vegetable oil, and combinations thereof.

33. A biodegradable microcapsule according to claim 27, wherein said plant-based protein hydrogel has been non-covalently modified by a non-covalent crosslinker selected from sodium tripolyphosphate, sodium hexametaphosphate, and phenolic compounds.

34. A biodegradable microcapsule according to claim 27, wherein said plant-based protein hydrogel has been covalently modified by a covalent cross-linker selected from genipin, epoxy compounds, glyceraldehyde, glutaraldehyde, formaldehyde, glyoxal, dialdehyde starch, microbial transglutaminase, and PolyCup® crosslinking resin, or combinations thereof.

35. A biodegradable microcapsule according to claim 27, wherein said coating is a metal coating, a silicon-based coating, a polymeric coating, a coacervate coating, or a mineral coating.

36. A biodegradable microcapsule according to claim 35, wherein said metal coating is a silver coating or a gold coating.

37. A biodegradable microcapsule according to claim 35, wherein said silicon-based coating is formed from a silicon-containing compound selected from the group consisting of tetraethyl orthosilicate, tetrapropyl orthosilicate, dimethyl diethoxysilane and tetramethyl orthosilicate and combinations thereof.

38. A biodegradable microcapsule according to claim 35, wherein said coacervate coating is formed from a polysaccharide selected from the group consisting of xanthan gum, gellan gum, and chitosan, and combinations thereof.

39. A biodegradable microcapsule according to claim 35, wherein said polymeric coating is formed from a polyisocyanate and an amine.

40. A biodegradable microcapsule according to claim 27, having a coating deposited on the covalently-modified or non-covalently modified plant-based protein hydrogel.

41. A biodegradable microcapsule according to claim 27, wherein the plant-based protein hydrogel comprises plant-based proteins having a protein secondary structure with at least 40% intermolecular β-sheet wherein the % intermolecular β-sheet content is measured by FTIR.

42. A biodegradable microcapsule according to claim 27, wherein the at least one fragrance material or flavour material is part of a fragrance or flavour and wherein at least 50% of the total fragrance or flavour encapsulated is present inside the microcapsule after incubation in hard water comprising 192 mg/l NaHCO₃, 120 mg/l CaSO₄·2H₂O, 120 mg/l MgSO₄, and 8 mg/l KCl at 37° C. for 72 hours, as determined by GC.

43. A biodegradable microcapsule according to claim 27, wherein the at least one fragrance material or flavour material is part of a fragrance or flavour and wherein at least 25% of the total fragrance or flavour encapsulated is present inside the microcapsule after incubation in the standard fabric conditioner formulation defined in Table 1 at 37° C. for 24 hours, as determined by GC.

44. A composition comprising a biodegradable microcapsule as claimed in claim 27 and an external phase.

45. A consumer product comprising a biodegradable microcapsule as claimed in claim 27.

46. A method of making a consumer product, comprising:
(a) preparing a biodegradable microcapsule according to the method of claim 1;
(b) mixing said biodegradable microcapsule with a consumer product formulation.

47. The use of a biodegradable microcapsule as claimed in claim 27 in a consumer product.
